(19) United States
(12) Patent Application Publication
(10) Pub. No.: US 2016/0367661 A1
(43) Pub. Date: Dec. 22, 2016

(54) NLRP6 Inflammasome Intestinal Epithelium Mucus Secretion

(71) Applicants: YALE UNIVERSITY, New Haven, CT (US); WEIZMANN INSTITUTE OF SCIENCE, Rehovot (IL); UNIVERSITY OF BRITISH COLUMBIA, Vancouver (CA)

(72) Inventors: Richard Flavell, Guilford, CT (US); Eran Elinav, Mazkeret Batya (IL); B. Brett Finley, Vancouver (CA)

(21) Appl. No.: 15/121,609

(22) PCT Filed: Feb. 27, 2015
(86) PCT No.: PCT/US15/18065

§ 371 Date: Aug. 25, 2016

Related U.S. Application Data
(60) Provisional application No. 61/945,447, filed on Feb. 27, 2014.

(51) Int. Cl. A61K 39/395; A61K 31/7088; A61K 45/06; A61K 38/02

(52) U.S. Cl. CPC A61K 39/395; A61K 38/02; A61K 31/7088; A61K 45/06

(57) ABSTRACT

The present invention provides a composition and method for treating or preventing a disease or disorder associated with intestinal microbiota.

### CROSS-REFERENCE TO RELATED APPLICATIONS

This application claims priority to U.S. Provisional Patent Application No. 61/945,447 filed Feb. 27, 2014, the contents of which are incorporated by reference herein in their entirety.

### BACKGROUND OF THE INVENTION

Inflammasomes are cytoplasmic multi-protein complexes that are expressed in various cell lineages and orchestrate diverse functions during homeostasis and inflammation. The complexes are composed of one of several NLR proteins, such as NLRP1, NLRP3, NLRC4 and NLRP6, which function as innate sensors of endogenous or exogenous stress or damage-associated molecular patterns. NLRP6 is a newly identified NLR protein that has been shown to participate in inflammasome signaling (Grenier et al., 2002, FEBS Letters, 530: 73-78) and to play critical roles in defense against infection, auto-inflammation and tumorigenesis (Anand et al., 2012, Nature, 488: 389-393; Chen et al., 2011, Journal of Immunology, 186: 7187-7194; Elinav et al., 2011b, Cell, 145: 745-757; Hu et al., 2013, Proceedings of the National Academy of Sciences of the United States of America, 110: 9862-9867; Normand et al., 2011, Proceedings of the National Academy of Sciences of the United States of America, 108: 9601-9606). NLRP6 is highly expressed in the intestinal epithelium (Chen et al., 2011, Journal of Immunology, 186: 7187-7194; Elinav et al., 2011b, Cell, 145: 745-757; Normand et al., 2011, Proceedings of the National Academy of Sciences of the United States of America, 108: 9601-9606), but the signal(s) and mechanisms leading to NLRP6 downstream effects remain elusive.

It is becoming clear that NLRP6 plays critical roles in maintaining intestinal homeostasis and a healthy intestinal microbiota. NLRP6 is essential for mucosal self-renewal and proliferation, rendering NLRP6 deficient mice more susceptible to intestinal inflammation and to chemically induced colitis as well as increased tumor development (Chen et al., 2011, Journal of Immunology, 186: 7187-7194; Normand et al., 2011, Proceedings of the National Academy of Sciences of the United States of America, 108: 9601-9606). Further contributing to intestinal health, NLRP6 participates in the steady-state regulation of the intestinal microbiota, partly through the basal secretion of IL-18 (Elinav et al., 2011b, Cell, 145: 745-757). NLRP6 deficiency leads to the development of a colitogenic microbiota that is intimately associated at the base of the colonic crypt, stimulating a pro-inflammatory immune response, ultimately leading to increased susceptibility to chemically induced colitis in NLRP6 deficient mice (Elinav et al., 2011b, Cell, 145: 745-757). However, the mechanisms by which the absence of a single inflammasome component leads to changes in intestinal microbial community composition and biogeographical distribution remain unknown.

Therefore, there is a need in the art for compositions and methods to modulate inflammasome function to treat and prevent diseases and disorders associated with altered microbiota. The present invention satisfies this unmet need.

### SUMMARY OF THE INVENTION

In one aspect, the present invention provides a composition for treating a disease or disorder associated with intestinal microbiota, where the composition comprises a modulator of intestinal epithelium mucin secretion. In one embodiment, the modulator of intestinal epithelium mucin secretion is a modulator of at least one component of the NLRP6 inflammasome. In one embodiment, the modulator of intestinal epithelium mucin secretion is a modulator of at least one component of the autophagy pathway. In one embodiment, the modulator of intestinal epithelium mucin secretion is a modulator of goblet cell mucin secretion.

In one embodiment, the modulator is at least one of the group consisting of a chemical compound, a protein, a peptide, a peptidomemetic, an antibody, a ribozyme, a small molecule chemical compound, a nucleic acid, a vector, an antisense nucleic acid molecule.

In one embodiment, the disease or disorder is at least one selected from the group consisting of a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, non-alcoholic fatty liver disease (NAFLD), and non-alcoholic steatohepatitis (NASH).

In one embodiment, the modulator of intestinal epithelium mucin secretion increases intestinal epithelium mucin secretion. In one embodiment, the modulator is a natural ligand expressed by intestinal microbiota.

The present invention also provides a method of treating a disease or disorder associated with intestinal microbiota, the method comprising increasing intestinal epithelium mucin secretion in a subject. In one embodiment, the method comprises administering to the subject a modulator of intestinal epithelium mucin secretion. In one embodiment, the modulator of intestinal epithelium mucin secretion is a modulator of at least one component of the NLRP6 inflammasome. In one embodiment, the modulator of intestinal epithelium mucin secretion is a modulator of at least one component of the autophagy pathway. In one embodiment, the modulator of intestinal epithelium mucin secretion is a modulator of goblet cell mucin secretion.

In one embodiment, the modulator is at least one of the group consisting of a chemical compound, a protein, a peptide, a peptidomemetic, an antibody, a ribozyme, a small molecule chemical compound, a nucleic acid, a vector, an antisense nucleic acid molecule.

In one embodiment, the disease or disorder is at least one selected from the group consisting of a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, non-alcoholic fatty liver disease (NAFLD), and non-alcoholic steatohepatitis (NASH).

In one embodiment, the modulator of intestinal epithelium mucin secretion increases intestinal epithelium mucin secretion. In one embodiment, the modulator is a natural ligand expressed by intestinal microbiota.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1J, depicts the results of experiments demonstrating that NLRP6 protects from enhanced enteric infection. WT and Nlrp6^{-/-} mice were infected with 10⁹ CFU of bioluminescent C. rodentium and analyzed on day 15 p.i., unless otherwise stated. (FIG. 1A) In vivo whole body bioluminescence imaging of WT and Nlrp6^{-/-} mice on day 9 p.i. show increased bacterial growth in Nlrp6^{-/-} mice. (FIG. 1B) Both luminal (fecal matter) and adherent (extensively washed colons) bacterial colonization is enhanced in Nlrp6^{-/-} mice. Results are pooled from two separate experiments, n=12-14 per group. Significance determined using the Mann-Whitney U-test. (**p<=0.0033; ****p<0.0001). (FIG. 1C) H&E stained distal colon sections from WT and Nlrp6^{-/-} mice show an increase in inflammation and crypt ulceration throughout the mucosa of Nlrp6^{-/-} mice. Magnification=5×, 10×; scale bar=200 μm. (FIG. 1D) Histopathology scores from distal colon tissues of Nlrp6^{-/-} and WT mice. Each bar represents one individual mouse and shows scores for damage to the submucosa, mucosa, surface epithelium and lumen, n=9 per group. (****p<0.0001) (FIG. 1E and FIG. 1F) Secretion of pro-inflammatory cytokines in the colon (FIG. 1E) and spleen (FIG. 1F) is unchanged between WT and Nlrp6^{-/-} mice. Results are pooled from two separate infections of WT and Nlrp6^{-/-} mice, n=13 and 14, respectively. (FIG. 1G) C. rodentium-specific colonic IgA and systemic IgG titers. Results are pooled from two separate experiments, n=9-13 per group. (FIG. 1H-FIG. 1J) Quantitative RT-PCR showing expression of IL-22 (FIG. 1H), Reg3β (FIG. 1I) and Reg3γ (FIG. 1J) relative to gapdh in the distal colon of WT and Nlrp6^{-/-} mice over the course of C. rodentium infection, n=4-9.

FIG. 2, comprising FIG. 2A through FIG. 2I, depicts the results of experiments demonstrating that inflammasome signaling is required for clearance of C. rodentium infection. WT, Asc^{-/-} and Caspase-1/11^{-/-} mice were infected with 10⁹ CFU of bioluminescent C. rodentium and analyzed on day 9 post infection. Representative images (FIG. 2A and FIG. 2F) and time course quantification (FIG. 2B and FIG. 2G) of in vivo whole body bioluminescence imaging shows elevated bacterial growth in the intestine of Asc^{-/-} (FIG. 2A and FIG. 2B) and Caspase-1/11^{-/-} mice (FIG. 2F and FIG. 2G). Ex vivo imaging of extensively washed colonic explants shows enhanced bacterial attachment to colons of Asc^{-/-} (FIG. 2C) and Caspase-1/11−/− (FIG. 2H) mice. Bacterial plating demonstrates a higher colonic and systemic colonization of Asc^{-/-} (FIG. 2D and FIG. 2E) and Caspase-1/11^{-/-} (FIG. 2I) mice.

FIG. 3, comprising FIG. 3A through FIG. 3D, depicts the results of experiments demonstrating that NLRP6 is expressed in goblet cells. (FIG. 3A) Analysis of NLRP6 expression in sorted colonic epithelial and hematopoietic (CD45+) cells. The purity of the sorted populations was analyzed by RT-qPCR using vil1 and ptprc as markers for epithelial and hematopoietic cells, respectively. NLRP6 expression closely mirrored that of colonic epithelial cells. (FIG. 3B-FIG. 3D) In situ hybridization with an NLRP6-specific probe, visible as black dots, with an H&E counter stain. The theca (housing all mucin-containing granules) within goblet cells is not stained with H&E and identified as un-stained circles allowing localization of goblet cells within the epithelium (outlined with black circles). (FIG. 3B) Representative localization of NLRP6 in a WT distal colon section, showing that staining is concentrated in the apical region of the epithelium. Magnifications demonstrate an enrichment of NLRP6 mRNA in proximity to goblet cells, seen as increased probe-binding to areas surrounding the theca of goblet cells. (FIG. 3C) As in FIG. 3B, but in Asc^{-/-} mice. (FIG. 3D) No nonspecific probe binding is seen in Nlrp6^{-/-} distal colon sections.

FIG. 4, comprising FIG. 4A through FIG. 4G, depicts the results of experiments demonstrating that NLRP6 inflammasome activity is required for goblet cell function and protection from C. rodentium invasiveness. (FIG. 4A) AB/PAS stained distal colon sections of WT, Asc^{-/-}, and Caspase-1/11^{-/-} mice showing the inner mucin layer (“i”) and goblet cells (asterisks). Magnification=400×; scale bar=50 (FIG. 4B) Quantification of inner mucus layer thickness in the distal colon. The inner mucus layer is absent in Nlrp6^{-/-} and Asc^{-/-} mice and significantly thinner in Caspase1/11^{-/-} mice, n=8, 4, and 5 mice, respectively (***p=<0.0001). (FIG. 4C) Quantification of goblet cell number in the distal colon. Nlrp6^{-/-} (***p=0.0001), Asc^{-/-} (***p=0.0001) and Caspase1/11^{-/-} (***p=0.0007) mice exhibit goblet cell hyperplasia, n=8, 4, and 5 mice, respectively. (FIG. 4D) Representative transmission electron microscopy images taken from colonic sections of WT and Nlrp6^{-/-} mice, n=4 mice per group. (FIG. 4E) Representative epifluorescence staining for mucus using the lectin UEA-1 with DAPI as a counter stain. The inner mucin layer is absent in Nlrp6^{-/-} mice. i=inner mucin layer. Original magnification=200×; scale bar=50 μm. (FIG. 4F) Representative immunostaining for the C. rodentium effector Tir and the mucus specific protein Muc2 in colon, with DAPI as a counter stain, in WT and Nlrp6^{-/-} mice at 7 days p.i. The inner mucus layer is visible in WT mice and is lacking in the Nlrp6^{-/-} mice. Magnification=200×; scale bar=50 μm, i=inner mucus layer. (FIG. 4G) In Nlrp6^{-/-} mice, C. rodentium appears to be more invasive, as shown by deeper penetration into the crypts, which often co-localizes with muc2.

FIG. 5, comprising FIG. 5A through FIG. 5F, depicts the results of experiments demonstrating that NLRP6 inflammasome is required for mucus granule exocytosis. (FIG. 5A) Representative AB/PAS stained colon sections showing the inner mucus layer (i) in WT mice. Nlrp6^{-/-} mice show the presence of mucus granules-like structures within the lumen (inset “a”). Scale bar=50 μm. (FIG. 5B) AB/PAS stained Nlrp6^{-/-} distal colon section showing accumulation of mucus granule-like structures in the lumen (arrowhead) and an increased number of large PAS+ goblet cells (asterisks). Scale bar=50 μm. (FIG. 5C) Representative immunostaining for the goblet cell specific protein, Clca3 with DAPI as a counter stain in distal colon sections. Arrowheads show diffuse staining of Clca3 in the WT lumen and punctate staining in the Nlrp6^{-/-} lumen. Representative transmission electron microscopy images (insets a and b) show intact mucus secretion by a goblet cell in WT and dysfunctional mucus granule exocytosis and the presence of granule-like structures in Nlrp6^{-/-} distal colon tissue. (FIG. 5D) Transmission electron microscopy image of the Nlrp6^{-/-} distal colon showing protrusion of mucus granules into the lumen without mucus secretion and intact mucus granules saturating the intestinal lumen, n=4 mice. (FIG. 5E) Representative scanning electron microscopy images of the distal colon of WT and Nlrp6^{-/-} mice, n=2 mice per group. Each experiment was repeated 3 times. A smooth intestinal epithelium is seen in WT mice. A large number of goblet cells with mucus granules protruding into the lumen (arrowheads) are seen in Nlrp6^{-/-} mice. (FIG. 5F) Enlarged scanning electron microscopy image of four goblet cells with protruding mucus granules into the Nlrp6^{-/-} intestinal lumen.

FIG. 6, comprising FIG. 6A through FIG. 6I, depicts the results of experiments demonstrating that NLRP6 is required for autophagosome formation in the intestinal epithelium. (FIG. 6A) Representative immunofluorescence image of WT (LC3, top panel) and NLRP6-deficient (LC3:Nlrp6^{-/-}, bottom panel) intestinal epithelium shows abrogated autophagy in the absence of NLRP6. Goblet cells are stained with the mucus specific protein Muc2, epithelial cell nuclei are indicated with DAPI. Formation of autophagosomes is visualized utilizing the LC3-GFP endogenously expressed protein. Scale=70 μm. (FIG. 6B) Magnification of intestinal epithelial cells showing WT goblet cells (Muc2 positive) active in the formation of autophagosomes, seen as punctate staining with the LC3-GFP endogenous protein co-localizing with Muc2 positive cells. (FIG. 6C) Quantitation of autophagosome formation through enumeration of LC3 puncta per 100 epithelial cells, n=5 mice per group (***p<0.0001). (FIG. 6D) Immunoblot analysis of total LC3-GFP, and p62 proteins in isolated intestinal epithelial cells from WT LC3-GFP transgenic mice and NLRP6-deficient GFP-LC3 transgenic mice. (FIG. 6E) LC3-GFP band intensities from FIG. 6D were quantified and normalized to actin band intensity, n=5 mice per group (**p=0.0067). (FIG. 6F) Immunoblot analysis of total endogenous LC3-I/II and p62 proteins in isolated intestinal epithelial cells of WT, Asc^{-/-} and Caspase-1/11^{-/-} mice. LC3-I and LC3-II denote the nonlipidated (cytosolic) and lipidated (autophagosome membrane) forms of LC3, respectively. (FIG. 6G) Accumulation of LC3-I in isolated epithelial cells from Nlrp6^{-/-} (**p=0.0015), Asc^{-/-} (**p=0.0013) and Caspase-1/11^{-/-} (**p=0.0025) mice, as shown by the fraction of LC3-I band density out of total LC3 band density. Data represent n=6 (WT, Asc^{-/-}) or n=4 (Caspase-1/11^{-/-}) mice. (FIG. 6H) Increased abundance of p62 in Nlrp6^{-/-} (*p=0.0349), Asc^{-/-} (ns, p=0.2115) and Caspase-1/11^{-/-} (*p=0.0284) mice, as shown by quantification of p62 band intensity. Data represent n=6 (WT, Asc^{-/-}) or n=4 (Caspase-1/11^{-/-}) mice. (FIG. 6I) Mitochondria were scored and enumerated in WT and Nlrp6^{-/-} intestinal epithelial cells as healthy, unhealthy and dense inclusion body containing, n=25 or 28 epithelial cells, respectively. Mitochondrial dysfunction was characterized in Nlrp6^{-/-} mice as a decrease in total healthy mitochondria (***p<0.0001) and an accumulation of unhealthy (***p<0.0001) and dense inclusion body-containing (***p=0.0002) mitochondria. Representative transmission electron microscopy images are shown (magnification=11500×) and healthy, unhealthy, and dense inclusion body containing mitochondria are depicted with corresponding asterisks within WT and Nlrp6^{-/-} intestinal epithelial cells.

FIG. 7, comprising FIG. 7A through FIG. 7D, depicts the results of experiments demonstrating that autophagy is required for goblet cell function and mucus secretion in the intestine. (FIG. 7A) Representative AB/PAS stained colon sections showing the inner mucus layer (i) in WT mice. Atg5 heterozygous mice show reduced production of the inner mucus layer and goblet cell hyperplasia (asterisk). Scale bar=50 (FIG. 7B) Quantification of inner mucus layer thickness in the distal colon. The inner mucus layer is significantly thinner in the Atg5^{+/-} distal colon, n=3 mice (***p=<0.0001). (FIG. 7C) Quantification of goblet cell number in the distal colon. Atg5^{+/-} mice exhibit goblet cell hyperplasia, n=3 mice (**p=0.0030). (FIG. 7D) Transmission electron microscopy image of Atg5^{+/-} showing reduced mucus secretion. Theca of WT mice fuse with surface of epithelium resulting in mucus granule shedding and release of contained mucins. Fusion and granule release is stalled in Atg5^{+/-} mice.

FIG. 8, comprising FIG. 8A through FIG. 8D, depicts the results of experiments demonstrating that NLRP6 is not required for the cellular response to infection. Quantitative RT-PCR showing expression of (FIG. 8A) IL-1β and (FIG. 8B) IL-18 relative to gapdh in the distal colon of WT and Nlrp6^{-/-} mice over the course of C. rodentium infection, n=4-9. Immunofluorescence analysis of (FIG. 8C) neutrophil (MPO-positive cells), and (FIG. 8D) lymphocyte (CD90.1 positive cells), infiltration of distal colon sections at day 7 and 15 post infection. Total cell number was determined by enumerating all cells per 40× field with 5 fields counted per tissue section. Results are averaged from a single experiment, n=4-6 mice per group (**p=0.0039).

FIG. 9, comprising FIG. 9A through FIG. 9E, depicts the results of experiments demonstrating that transmissible colitogenic gut microbiota of NLRP6 deficient mice is not the cause of abnormal goblet cell function and mucus secretion. (FIG. 9A) Quantitative RT-PCR results of mud-4, tff3, and relmb (*p=0.0363), relative to gapdh expression in the distal colon of WT and Nlrp6^{-/-} mice. Results are representative of two independent experiments, n=4 per group. (FIG. 9B) Representative AB/PAS stained colon sections showing the inner mucus layer (i) in WT singly housed mice, WT co-housed mice with Nlrp6^{-/-} or Asc^{-/-} mice, and Nlrp6^{-/-} and Asc^{-/-} mice cohoused with WT mice. Brackets indicate co-housing partners. WT mice show normal goblet cell number (asterisks) and inner mucus layer (i), independent of housing conditions. Co-housing Nlrp6^{-/-} and Asc^{-/-} mice with WT mice does not rescue the defect in mucus production, and goblet cell hyperplasia is maintained. Scale bar=50 μm. (FIG. 9C) Quantification of inner mucus layer thickness in the distal colon. The inner mucus layer of singly-housed WT mice is similar to WT mice co-housed with Nlrp6^{-/-} mice or Asc^{-/-} mice, n=3 mice per group. (FIG. 9D and FIG. 9E) Co-housing of WT mice with Nlrp6^{-/-} (FIG. 9D) or Asc^{-/-} (FIG. 9E) mice does not result in goblet cell hyperplasia as exhibited by Nlrp6^{-/-} (FIG. 9D, **p=0.0040) or Asc^{-/-} (FIG. 9E, *p=0.0279) mice, n=3 mice per group.

FIG. 10, comprising FIG. 10A through FIG. 10C, depicts the results of experiments demonstrating that goblet cell function and mucus secretion is independent of signaling through IL-1R and IL-18. (FIG. 10A) Representative AB/PAS stained colon sections showing the inner mucus layer (i) in WT, IL-1R^{-/-} and IL-18^{-/-} mice. Scale bar=50 μm. (FIG. 10B) Quantification of inner mucus layer thickness in the distal colon. The inner mucus layer of WT mice is similar to IL-1R and IL-18 deficient mice, n=4-6 mice per group. (FIG. 10C) Quantification of goblet cell number in the distal colon. Goblet cell number is unchanged with IL-1R or IL-18 deficiency, n=4-6 mice per group.

FIG. 11, comprising FIG. 11A through FIG. 11I, depicts the results of experiments demonstrating that members of the NLRP6 inflammasome complex, Caspase-1/11 and ASC, are required for mucus exocytosis. Representative transmission electron microscopy images of the distal colon of WT (FIG. 11A), Caspase-1/11^{-/-} (FIG. 11D) and Asc^{-/-} (FIG. 11G) mice. Lack of visible mucus secretion in Caspase-1/11− (FIG. 11D) and Asc− (Figure G) deficient distal colon, n=2-4 per group. Enlarged images demonstrate emptying of the theca by WT goblet cells (FIG. 11B) and smaller theca with stalled secretion in Caspase-1/11^{-/-} (FIG. 11E) goblet cells. Representative scanning electron microscopy images of the surface of the distal colon of WT (FIG. 11C), Caspase-1/11^{-/-} (FIG. 11F), and Asc^{-/-} (FIG. 11H) mice, n=2 per group. Scale=20 (FIG. 11I) Enlarged image showing protruding mucus granules (arrow heads) in Asc^{-/-} mice (FIG. 11H), scale=20

FIG. 12 is a graph depicting the results of a high purity sorting experiment during Citrobacter infection at day 10 of infection. Colonic cells were sorted at the peak of Citrobacter infection to produce a high purity hematopoietic cell and epithelial cell subsets. Sorted epithelial cells are entirely devoid of ptprc⁺ (CD45) hematopoietic cells, while hematopoietic cells feature a low level epithelial contamination (manifesting as a 1:100 villin expression as compared to the epithelial cell expression levels). NLRP6 levels during infection closely mirrored the epithelial expression pattern in both the epithelial cell compartment and hematopoietic compartment, demonstrating that colonic epithelial cells are the near exclusive intestinal contributor to NLRP6 expression.

FIG. 13 is a graph depicting the Citrobacter burden (CFU) in WT littermates compared to N6 mice at 17 days post infection. It was observed that the WT littermate controls clear Citrobacter from the colonic wall significantly faster (Mann-Whitney test, **P=0.0087) than NLRP6 deficient mice, the same observation that was made for purchased WT mice (FIG. 3A).

FIG. 14 is a set of graphs depicting the results of experiments examining burden (CFU) for colon luminal bacteria (top) and colon adherent bacteria (bottom) for wildtype (WT), wildtype cohoused with NLRP6^{-/-} (WT-co), and NLRP6^{-/-} mice. It was observed that NLPR6 susceptibility to Citrobacter infection is at least partially independent of the altered microbiota (see no difference between cohoused WT and WT)

### DETAILED DESCRIPTION

The present invention is related to the discovery of the role of the NLRP6 inflammasome in regulating mucin secretion and autophagy in the intestinal epithelium, which are crucial for maintaining an intestinal barrier to microbial and pathogen penetration.

In one aspect, the present invention provides compositions and methods to treat or prevent a disease or disorder associated with intestinal microbiota. For example, in certain embodiments, the invention provides a composition and method for the treatment or prevention of an intestinal infection. In one embodiment, the invention provides a composition and method for the treatment or prevention of a disease or disorder associated with microbial dysbiosis.

In one aspect, the present invention provides a method to diagnose a disease or disorder associated with intestinal microbiota. For example, in one embodiment, one or more components of the NLRP6 inflammasome, mucin secretion pathway, or autophagy pathway are used as diagnostic markers.

It is described herein that the NLRP6 inflammasome simultaneously influences intestinal barrier function and microbial homeostasis, through regulation of goblet cell mucus secretion. Deficiency in the expression or activity of one or more components of the NLRP6 inflammasome results in impairment of mucin granule exocytosis and resultant mucus layer formation, leading to increased susceptibility to enteric infection. Mechanistically, NLRP6 deficiency leads to abrogation of autophagy, providing a link between inflammasome activity, autophagy, mucus exocytosis, and antimicrobial barrier function.

Therefore, in certain aspects, the present invention relates to modulating the mucin secretion of the intestinal epithelium, thereby treating or preventing a disease or disorder associated with intestinal microbiota. For example, in certain embodiments, the invention relates to compositions and methods for increasing the expression or activity of one or more components of the NLRP6 inflammasome, increasing the formation and function of goblet cell autophagosomes, and increasing goblet cell mucin secretion.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient. The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of pathology, for the purpose of diminishing or eliminating those signs or symptoms.

As used herein, “treating a disease or disorder” means reducing the severity and/or frequency with which a sign or symptom of the disease or disorder is experienced by a patient. Disease and disorder are used interchangeably herein.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “microbiota” is used to refer to the community of microbes that occupy the digestive tract of a subject.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, a “modulator of a component of the NLRP6 inflammasome” is a compound that modifies the expression, activity or biological function of the component of the NLRP6 inflammasome as compared to the expression, activity or biological function of the component of the NLRP inflammasome in the absence of the modulator.

As used herein, the term “antagonist of a component of the NLRP6 inflammasome” refers to a compound that inhibits, reduces, or blocks the biological activity or expression of the component of the NLRP6 inflammasome. Suitable antagonists include, but are not limited to, antibodies and antibody fragments, polypeptides including fragments of the component of the NLRP6 inflammasome, small organic compounds, nucleic acids, natural ligands, and microbe component natural ligands.

As used herein, the term “agonist of a component of the NLRP6 inflammasome” refers to a compound that increases the biological activity or expression of the component of the NLRP6 inflammasome. Suitable agonists include, but are not limited to, antibodies and antibody fragments, polypeptides, small organic compounds, nucleic acids, natural ligands, and microbe component natural ligands.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is partly based upon the discovery of the role of the NLRP6 inflammasome in regulating autophagy and mucin secretion in the intestinal epithelium, which are crucial for maintaining an intestinal barrier to microbial and pathogen penetration.

In one aspect, the present invention provides a composition and method to treat or prevent a disease or disorder associated with intestinal microbiota. In one embodiment, the invention provides a composition and method for the treatment or prevention of an intestinal infection. In one embodiment, the invention provides a composition and method for the treatment or prevention of a disease or disorder associated with microbial dysbiosis.

It is described herein that NLRP6 control of mucus secretion directly affects its ability to regulate intestinal and microbial homeostasis while creating a protective niche from enteric pathogens. Thus, reduced expression or defective components of the inflammasome signaling pathway leads to abrogated mucus secretion characterized by protruding mucin granules, that rather than fusing into the apical basement membrane and releasing their content, are sloughed off into the intestinal lumen in their entirety. The lack of mucus secretion and inability to form an adherent, continuous inner mucus layer would allow for close microbe-epithelium interactions, leaving a subject susceptible to infection, as well as other consequences associated with dysbiotic microbiota.

The present invention is partly based upon the discovery that NLRP6 is present in the goblet cells of the intestinal epithelium, and that NLRP6 regulates goblet cell mucin secretion and autophagy. It is demonstrated herein that goblet cells, previously regarded as passive contributors to the formation of the biophysical protective mucosal layers, are actually active, regulatory hubs integrating signals from the host and its environment as an integral component of the innate immune response.

In one embodiment, the invention provides a composition for the treatment or prevention of a disease or disorder associated with intestinal microbiota. In one embodiment, the composition comprises a modulator of intestinal epithelium mucin secretion.

In one embodiment, the composition comprises a modulator of the expression or activity of one or more components of the NLRP6 inflammasome. For example, in one embodiment, the modulator increases the expression or activity of one or more components of the NLRP6 inflammasome. The one or more components of the NLRP6 inflammasome, include, but are not limited to NLRP6, ASC, and Caspase-1, and Caspase-11.

In one embodiment, the composition comprises a modulator of one or more components of the autophagy pathway. For example, in one embodiment, the modulator increases the expression or activity of one or more components of the autophagy pathway. In one embodiment, the composition comprises a modulator of one or more components of an autophagosome. Exemplary components of the autophagy pathway include, but are not limited to, Beclin-1, Vps34, ULK1, WIPI-1, FIP200, LC3, and any of the autophagy related proteins (Atg), including but not limited to, Atg2, Atg 3, Atg4, Atg5, Atg7, Atg8, Atg9, Atg10, Atg12, Atg13, Atg14, Atg16, and the like.

The present invention provides a method for treating or preventing a disease or disorder associated with intestinal microbiota in a subject in need. In one embodiment, the method treats or prevents an infection, including for example a bacterial infection, viral infection, fungal infection, and the like. It is found herein that increasing the level of expression or activity of the inflammasome or autophagy pathway can restore, maintain or improve the intestinal barrier, thereby making a subject less susceptible to infection.

In one embodiment, the method treats or prevents a disease or disorder associated with microbial dysbiosis in a subject in need. For example, in certain instances, diminished inflammasome expression or activity may lead to an altered microbiota in a subject, which in turn can lead to a wide variety of diseases and disorders including, but not limited to inflammatory bowel disease, celiac disease, colitis, intestinal hyperplasia, cancer, metabolic syndrome, obesity, rheumatoid arthritis, liver disease, hepatic steatosis, fatty liver disease, non-alcoholic fatty liver disease (NAFLD), and non-alcoholic steatohepatitis (NASH).

As described herein, such diseases and disorders are caused, at least in part, by a reduction in autophagy and mucin secretion by the intestinal epithelium.

Therefore, in certain aspects, the present invention relates to increasing the expression or activity of one or more components of the NLRP6 inflammasome, increasing one or more components of the autophagy pathway in goblet cells, increasing the formation and function of goblet cell autophagosomes, and increasing goblet cell mucin secretion.

In one aspect, the present invention provides a method to diagnose a disease or disorder associated with intestinal microbiota. For example, in one embodiment, one or more components of the NLRP6 inflammasome, mucin secretion pathway, or autophagy pathway are used as diagnostic markers.

### Therapeutic Modulator Compositions

In various embodiments, the present invention includes modulator compositions and methods of preventing and treating a disease or disorder associated with intestinal microbiota. In various embodiments, the modulator compositions and methods of treatment of the invention modulate the level or activity of a gene, or gene product. In some embodiments, the modulator composition of the invention is an activator that increases the level or activity of a gene, or gene product.

It will be understood by one skilled in the art, based upon the disclosure provided herein, that modulating a gene, or gene product, encompasses modulating the level or activity of a gene, or gene product, including, but not limited to, modulating the transcription, translation, splicing, degradation, enzymatic activity, binding activity, or combinations thereof. Thus, modulating the level or activity of a gene, or gene product includes, but is not limited to, modulating transcription, translation, degradation, splicing, or combinations thereof, of a nucleic acid; and it also includes modulating any activity of polypeptide gene product as well.

In one embodiment, the modulator increases the expression or activity of a gene or gene product by increasing production of the gene or gene product, for example by modulating transcription of the gene or translation of the gene product. In one embodiment, the modulator increases the expression or activity of a gene or gene product by providing exogenous gene or gene product. In one embodiment, the modulator increases the expression or activity of a gene or gene product by inhibiting the degradation of the gene or gene product. For example, in one embodiment, the modulator decreases the ubiquitination, proteosomal degradation, or proteolysis of a gene product. In one embodiment, the modulator increases the stability or half-life of a gene product.

In various embodiments, the modulated gene, or gene product, is one or more components of the NLRP6 inflammasome. For example, it is described herein that the NLRP6 inflammasome regulates the mucin secretion of the intestinal epithelium which provides a protective barrier between intestinal microbiota and epithelium. In one embodiment, the gene or gene product is one or more components of the NLRP6 inflammasome, including, but not limited to NLRP6, ASC, Caspase-1, and Caspase-11.

In various embodiments, the modulated gene or gene product is one or more components of the mucin secretion pathway.

In various embodiments, the modulated gene or gene product is one or more components of the autophagy pathway. For example, in one embodiment, the modulated gene or gene product is one or more components that play a role in autophagosome formation and activity. It is described herein that autophagy in the intestinal epithelium is responsible for the release or secretion of mucin granules. In one embodiment, the gene or gene product is one or more components of the autophagy pathway, including, but not limited to Beclin-1, Vps34, ULK1, WIPI-1, FIP200, LC3, and any of the autophagy related proteins (Atg), including but not limited to, Atg2, Atg 3, Atg4, Atg5, Atg7, Atg 8, Atg9, Atg10, Atg12, Atg13, Atg14, Atg16, and the like.

Modulation of a gene, or gene product, can be assessed using a wide variety of methods, including those disclosed herein, as well as methods known in the art or to be developed in the future. That is, the routineer would appreciate, based upon the disclosure provided herein, that modulating the level or activity of a gene, or gene product, can be readily assessed using methods that assess the level of a nucleic acid encoding a gene product (e.g., mRNA), the level of polypeptide gene product present in a biological sample, the activity of polypeptide gene product present in a biological sample, or combinations thereof.

The modulator compositions and methods of the invention that modulate the level or activity of a gene, or gene product, include, but should not be construed as being limited to, a chemical compound, a protein, a peptide, a peptidomemetic, an antibody, a ribozyme, a small molecule chemical compound, a nucleic acid, a vector, an antisense nucleic acid molecule (e.g., siRNA, miRNA, etc.), or combinations thereof. One of skill in the art would readily appreciate, based on the disclosure provided herein, that a modulator composition encompasses a chemical compound that modulates the level or activity of a gene, or gene product. Additionally, a modulator composition encompasses a chemically modified compound, and derivatives, as is well known to one of skill in the chemical arts.

In one embodiment, the modulator composition of the present invention is an antagonist, which inhibits the expression, activity, or biological function of a gene or gene product. For example, in certain embodiments, the modulator of the present invention is an antagonist of at least one component of the NLRP6 inflammasome, mucin secretion pathway, or autophagy pathway.

In one embodiment, the modulator composition of the present invention is an agonist, which increases the expression, activity, or biological function of a gene or gene product. For example, in certain embodiments, the modulator of the present invention is an agonist of at least one component of the NLRP6 inflammasome, mucin secretion pathway, or autophagy pathway.

Further, one of skill in the art would, when equipped with this disclosure and the methods exemplified herein, appreciate that modulators include such modulators as discovered in the future, as can be identified by well-known criteria in the art of pharmacology, such as the physiological results of modulation of the genes, and gene products, as described in detail herein and/or as known in the art. Therefore, the present invention is not limited in any way to any particular modulator composition as exemplified or disclosed herein; rather, the invention encompasses those modulator compositions that would be understood by the routineer to be useful as are known in the art and as are discovered in the future.

In certain embodiments, the modulator composition comprises a peptide, small molecule, or the like, from a naturally occurring source. For example, in one embodiment, the modulator composition comprises a natural ligand or microbe component natural ligand, which may be expressed on the surface of a microorganism of the gut or secreted by a microorganism of the gut.

In certain embodiments, a natural ligand or microbe component natural ligand of gut microbiota modulates the expression or activity of one or more components of the NLRP6 inflammasome. In one embodiment, a natural ligand or microbe component natural ligand is an antagonist of one or more components of the NLRP6 inflammasome, thereby reducing the expression or activity of the one or more components of the NLRP inflammasome. In one embodiment, a natural ligand or microbe component natural ligand is an agonist of one or more components of the NLRP6 inflammasome, thereby increasing the expression or activity of the one or more components of the NLRP inflammasome.

In certain embodiments, a natural ligand or microbe component natural ligand of gut microbiota modulates the expression or activity of one or more components of the autophagy pathway. In one embodiment, a natural ligand or microbe component natural ligand is an antagonist of one or more components of the autophagy pathway, thereby reducing the expression or activity of the one or more components of the autophagy pathway. In one embodiment, a natural ligand or microbe component natural ligand is an agonist of one or more components of the autophagy pathway, thereby increasing the expression or activity of the one or more components of the autophagy pathway.

Further methods of identifying and producing modulator compositions are well known to those of ordinary skill in the art, including, but not limited, obtaining a modulator from a naturally occurring source (i.e., Streptomyces sp., Pseudomonas sp., Stylotella aurantium). Alternatively, a modulator can be synthesized chemically. Further, the routineer would appreciate, based upon the teachings provided herein, that a modulator composition can be obtained from a recombinant organism. Compositions and methods for chemically synthesizing modulators and for obtaining them from natural sources are well known in the art and are described in the art.

One of skill in the art will appreciate that a modulator can be administered as a small molecule chemical, a polypeptide, a peptide, an antibody, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a peptide or a nucleic acid encoding a peptide that is modulator of a gene, or gene product, associated with a disease or disorder associated with intestinal microbiota. For example, the invention includes a peptide or a nucleic acid encoding a peptide that is one or more components of the NLRP6 inflammasome or autophagy pathway (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

### Peptides

In one embodiment, the composition of the present invention comprises one or more peptides. For example, in one embodiment, a peptide of the composition comprises an amino acid sequence of one or more components of the NLRP6 inflammasome. In one embodiment, a peptide of the composition of the invention comprises an amino acid sequence of one or more components of the autophagy pathway. In one embodiment, a peptide of the composition increases the expression or activity of one or more components of the NLRP6 inflammasome or autophagy pathway.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

The peptides of the invention may be prepared by standard chemical or biological means of peptide synthesis. Biological methods include, without limitation, expression of a nucleic acid encoding a peptide in a host cell or in an in vitro translation system.

Included in the invention are nucleic acid sequences that encode the peptide of the invention. In one embodiment, the invention includes nucleic acid sequences encoding the amino acid sequence of one or more components of the NLRP6 inflammasome and one or more components of the autophagy pathway. Accordingly, subclones of a nucleic acid sequence encoding a peptide of the invention can be produced using conventional molecular genetic manipulation for subcloning gene fragments, such as described by Sambrook et al., Molecular Cloning: A Laboratory Manual, Cold Springs Laboratory, Cold Springs Harbor, New York (2012), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for a particular activity.

Combined with certain formulations, such peptides can be effective intracellular agents. However, in order to increase the efficacy of such peptides, the one or more peptides of the invention can be provided a fusion peptide along with a second peptide which promotes “transcytosis”, e.g., uptake of the peptide by epithelial cells. To illustrate, the one or more peptides of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the one or more peptides can be provided a fusion polypeptide with all or a portion of the antenopedia III protein.

### Nucleic Acids

In one embodiment, the composition of the invention comprises one or isolated nucleic acids. For example, in one embodiment, the one or more isolated nucleic acids encodes one or more components of the NLRP6 inflammasome. In one embodiment, the one or more isolated nucleic acids encodes one or more components of the autophagy pathway. In one embodiment, the one or more nucleic acids encodes a peptide which increases the expression or activity of one or more components of the NLRP6 inflammasome or autophagy pathway.

In certain embodiments, a peptide corresponding to one or more components of the NLRP6 inflammasome or autophagy pathway is expressed from the one or more nucleic acids in a cell in vivo or in vitro using known techniques.

Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

The desired nucleic acid encoding one or more components of the NLRP6 inflammasome or autophagy pathway can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In one embodiment, the promoter or enhancer specifically directs expression of the one or more components of the NLRP6 inflammasome or autophagy pathway in the intestinal epithelium. For example, in one embodiment, the promoter or enhancer specifically directs expression of the one or more components of the NLRP6 inflammasome or autophagy pathway in a goblet cell.

In order to assess the expression of the desired polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82).

Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

In one embodiment, the composition of the invention comprises in vitro transcribed (IVT) RNA encoding one or more components of the NLRP6 inflammasome or autophagy pathway. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is one or more components of the NLRP6 inflammasome or autophagy pathway of the present invention.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

In one embodiment, the composition of the present invention comprises a modified nucleic acid encoding one or more components of the NLRP6 inflammasome or autophagy pathway described herein. For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low immunogenicity, and enhanced translation. Nucleoside-modified mRNA useful in the present invention is further described in U.S. Pat. No. 8,278,036, which is incorporated by reference herein in its entirety.

### Therapeutic Methods

The present invention also provides therapeutic methods for a disease or disorder associated with intestinal microbiota by modulating NLRP6 inflammasome activity, mucin secretion, autophagy, or a combination thereof.

For example, in one embodiment, the method treats or prevents an infection, for example a bacterial infection or viral infection. In one embodiment, the method treats or prevents a disease or disorder associated with microbial dysbiosis.

In various embodiments, the diseases and disorders treatable by the methods of the invention include, but are not limited to: bacterial infection, viral infection, inflammatory bowel disease, celiac disease, colitis, intestinal hyperplasia, cancer, metabolic syndrome, obesity, rheumatoid arthritis, liver disease, hepatic steatosis, fatty liver disease, non-alcoholic fatty liver disease (NAFLD), or non-alcoholic steatohepatitis (NASH).

It will be appreciated by one of skill in the art, when armed with the present disclosure including the methods detailed herein, that the invention is not limited to treatment of a disease or disorder associated with intestinal microbiota that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with intestinal microbiota, in that a modulator composition, as discussed previously elsewhere herein, can be administered to a subject prior to the onset of the disease or disorder, thereby preventing the disease or disorder. The preventive methods described herein also include the treatment of a subject that is in remission for the prevention of a recurrence an inflammatory disease or disorder associated with an altered microbiota.

One of skill in the art, when armed with the disclosure herein, would appreciate that the prevention of a disease or disorder associated with intestinal microbiota, encompasses administering to a subject a modulator composition as a preventative measure against the development of, or progression of a disease or disorder associated with intestinal microbiota. As more fully discussed elsewhere herein, methods of modulating the level or activity of a gene, or gene product, encompass a wide plethora of techniques for modulating not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases, disorders and pathologies associated with intestinal microbiota, where modulating the level or activity of a gene, or gene product treats or prevents the disease or disorder. Various methods for assessing whether a disease is associated with intestinal microbiota are known in the art. Further, the invention encompasses treatment or prevention of such diseases discovered in the future.

The invention encompasses administration of a modulator of a gene, or gene product. To practice the methods of the invention; the skilled artisan would understand, based on the disclosure provided herein, how to formulate and administer the appropriate modulator composition to a subject. The present invention is not limited to any particular method of administration or treatment regimen.

In one embodiment, the method comprises administering to the subject in need an effective amount of a composition that increases the expression or activity of one or more components of the NLRP6 inflammasome. For example, in one embodiment, the method comprises administering to the subject in need an effective amount of a composition that increases the expression or activity of one or more components of the NLRP6 inflammasome including but not limited to NLRP6, ASC, and caspase 1/11.

In one embodiment, the method comprises administering to the subject in need an effective amount of a composition that increases the expression or activity of one or more components of the autophagy pathway. For example, in one embodiment, the method comprises administering to the subject in need an effective amount of a composition that increases the expression or activity of one or more components of the autophagy pathway including but not limited to Atg5 and LC3.

In one embodiment, the method comprises increasing the expression or activity of the one or more components of the NLRP6 inflammasome or autophagy pathway in the intestinal epithelium of the subject. For example in one embodiment, the method comprises increasing the expression or activity of the one or more components of the NLRP6 inflammasome or autophagy pathway in a goblet cell of the subject.

In one embodiment, the method comprises contacting the intestinal epithelium of a subject with an effective amount of a composition that increases the expression or activity of one or more components of the NLRP6 inflammasome or autophagy pathway. For example, in one embodiment, the method comprises contacting a goblet cell of a subject with an effective amount of a composition that increases the expression or activity of one or more components of the NLRP6 inflammasome or autophagy pathway.

One of skill in the art will appreciate that the modulators of the invention can be administered singly or in any combination. Further, the modulators of the invention can be administered singly or in any combination in a temporal sense, in that they may be administered concurrently, or before, and/or after each other. One of ordinary skill in the art will appreciate, based on the disclosure provided herein, that the modulator compositions of the invention can be used to prevent or to treat a disease or disorder associated with intestinal microbiota, and that a modulator composition can be used alone or in any combination with another modulator to effect a therapeutic result. In various embodiments, any of the modulators of the invention described herein can be administered alone or in combination with other modulators of other molecules associated a disease or disorder associated with intestinal microbiota. Non-limiting examples of modulators that can be used in combination with the modulators and methods of the invention include: steroids, glucocorticoid steroids, corticosteroids, anti-biotics, anti-virals, non-steroidal anti-inflammatory drugs, and antibodies that specifically bind to pro-inflammatory mediators and/or their receptors, including α-IL-1, α-TNFα, α-IFNγ, α-TNFβ, α-IL4, α-IL5, α-IL6, α-IL10, and α-IL13.

In certain embodiments, the method comprises administering the composition of the invention along with an effective amount of an antibiotic composition. The type and dosage of the administered antibiotic will vary widely, depending upon the nature of the disease or disorder, the character of subject's microbiota, the subject's medical history, the frequency of administration, the manner of administration, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In various embodiments, the administered antibiotic is at least one of lipopeptide, fluoroquinolone, ketolide, cephalosporin, amikacin, gentamicin, kanamycin, neomycin, netilmicin, paromomycin, streptomycin, tobramycin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime cefluprenam, cefoselis, cefozopran, cefpirome, cefquinome, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftioxide, cefuracetime, imipenem, primaxin, doripenem, meropenem, ertapenem, flumequine, nalidixic acid, oxolinic acid, piromidic acid pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, azithromycin, erythromycin, clarithromycin, dirithromycin, roxithromycin, telithromycin, amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, ticarcillin, sulfamethizole, sulfamethoxazole, sulfisoxazole, trimethoprim-sulfamethoxazole, demeclocycline, doxycycline, minocycline, oxytetracycline, tetracycline, linezolid, clindamycin, metronidazole, vancomycin, vancocin, mycobutin, rifampin, nitrofurantoin, chloramphenicol, or derivatives thereof.

### Gene Therapy

Contacting cells in an individual with a functionally equivalent gene, gene product or a therapeutic agent that increases the expression or activity of one or more components of the NLRP6 inflammasome or autophagy pathway can inhibit or delay the onset of one or more symptoms of a disease or disorder associated with intestinal microbiota.

According to the present invention, a method is also provided of supplying protein to a cell which carries a mutant gene associated with diminished NLRP6 inflammasome or autophagy pathway activity. Supplying protein to a cell should allow normal functioning of the recipient cells. In certain embodiments, the protein supplied to the cell is a wild-type protein. The wild-type gene or a part of the gene may be introduced into the cell in a vector such that the gene remains extrachromosomal. In such a situation, the gene will be expressed by the cell from the extrachromosomal location. More preferred is the situation where the wild-type gene or a part thereof is introduced into the mutant cell in such a way that it recombines with the endogenous mutant gene present in the cell. Such recombination requires a double recombination event which results in the correction of the gene mutation. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner.

As generally discussed above, a gene or gene fragment, where applicable, may be employed in gene therapy methods in order to increase the amount of the expression products of the wild type gene even in those persons in which the wild type gene is expressed at a “normal” level, but the gene product is not fully functional.

“Gene therapy” includes both conventional gene therapy where a lasting effect is achieved by a single treatment, and the administration of gene therapeutic agents, which involves the one time or repeated administration of a therapeutically effective DNA or mRNA. Oligonucleotides can be modified to enhance their uptake, e.g. by substituting their negatively charged phosphodiester groups by uncharged groups. Components of the NLRP6 inflammasome or autophagy pathway of the present invention can be delivered using gene therapy methods, for example locally in the intestinal epithelium or systemically (e.g., via vectors that selectively target specific tissue types, for example, tissue-specific adeno-associated viral vectors). In some embodiments, primary cells (such as lymphocytes or stem cells) from the individual can be transfected ex vivo with a gene encoding any of the fusion proteins of the present invention, and then returning the transfected cells to the individual's body.

Gene therapy methods are well known in the art. See, e.g., WO96/07321 which discloses the use of gene therapy methods to generate intracellular antibodies. Gene therapy methods have also been successfully demonstrated in human patients. See, e.g., Baumgartner et al., Circulation 97: 12, 1114-1123 (1998), Fatham, C. G. ‘A gene therapy approach to treatment of autoimmune diseases’, Immun. Res. 18:15-26 (2007); and U.S. Pat. No. 7,378,089, both incorporated herein by reference. See also Bainbridge J W B et al. “Effect of gene therapy on visual function in Leber's congenital Amaurosis”. N Engl J Med 358:2231-2239, 2008; and Maguire A M et al. “Safety and efficacy of gene transfer for Leber's Congenital Amaurosis”. N Engl J Med 358:2240-8, 2008.

There are two major approaches for introducing a nucleic acid encoding a peptide or protein (optionally contained in a vector) into a patients cells; in vivo and ex vivo. For in vivo delivery, in certain instances, the nucleic acid is injected directly into the patient, usually at the site where the protein is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. Commonly used vectors for ex vivo delivery of the gene are retroviral and lentiviral vectors.

Gene therapy would be carried out according to generally accepted methods, for example, as described by Friedman et al., 1991, Cell 66:799-806 or Culver, 1996, Bone Marrow Transplant 3:S6-9; Culver, 1996, Mol. Med. Today 2:234-236. In one embodiment, cells from a patient would be first analyzed by the diagnostic methods known in the art, to ascertain the production and mutational status of a protein which is a component of the NLRP6 inflammasome or autophagy pathway. A virus or plasmid vector (see further details below), containing a copy of the gene or a functional equivalent thereof linked to expression control elements and capable of replicating inside the cells, is prepared. The vector may be capable of replicating inside the cells. Alternatively, the vector may be replication deficient and is replicated in helper cells for use in gene therapy. Suitable vectors are known, such as disclosed in U.S. Pat. No. 5,252,479 and PCT published application WO 93/07282 and U.S. Pat. Nos. 5,691,198; 5,747,469; 5,436,146 and 5,753,500. The vector is then injected into the patient. If the transfected gene is not permanently incorporated into the genome of each of the targeted cells, the treatment may have to be repeated periodically.

Gene transfer systems known in the art may be useful in the practice of the gene therapy methods of the present invention. These include viral and nonviral transfer methods. A number of viruses have been used as gene transfer vectors or as the basis for repairing gene transfer vectors, including papovaviruses (e.g., SV40, Madzak et al., 1992, J. Gen. Virol. 73:1533-1536), adenovirus (Berkner, 1992;Curr. Topics Microbiol. Immunol. 158:39-66), vaccinia virus (Moss, 1992, Current Opin. Biotechnol. 3:518-522; Moss, 1996, PNAS 93:11341-11348), adeno-associated virus (Russell and Hirata, 1998, Mol. Genetics 18:325-330), herpesviruses including HSV and EBV (Fink et al., 1996, Ann. Rev. Neurosci. 19:265-287), lentiviruses (Naldini et al., 1996, PNAS 93:11382-11388), Sindbis and Semliki Forest virus (Berglund et al., 1993, Biotechnol. 11:916-920), and retroviruses of avian (Petropoulos et al., 1992, J. Virol. 66:3391-3397), murine (Miller, 1992, Hum. Gene Ther. 3:619-624), and human origin (Shimada et al., 1991; Helseth et al., 1990; Page et al., 1990; Buchschacher and Panganiban, 1992, J. Virol. 66:2731-2739). Most human gene therapy protocols have been based on disabled murine retroviruses, although adenovirus and adeno-associated virus are also being used.

Nonviral gene transfer methods known in the art include chemical techniques such as calcium phosphate coprecipitation; mechanical techniques, for example microinjection; membrane fusion-mediated transfer via liposomes; and direct DNA uptake and receptor-mediated DNA transfer (Curiel et al., 1992, Am. J. Respir. Cell. Mol. Biol 6:247-252). Viral-mediated gene transfer can be combined with direct in vitro gene transfer using liposome delivery, allowing one to direct the viral vectors to the tumor cells and not into the surrounding non-dividing cells. Injection of producer cells would then provide a continuous source of vector particles. This technique has been approved for use in humans with inoperable brain tumors.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged. For other techniques for the delivery of adenovirus based vectors see U.S. Pat. Nos. 5,691,198; 5,747,469; 5,436,146 and 5,753,500.

Liposome/DNA complexes have been shown to be capable of mediating direct in vivo gene transfer. While in standard liposome preparations the gene transfer process is nonspecific, localized in vivo uptake and expression have been reported in tumor deposits, for example, following direct in situ administration.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes a protein, expression will produce the protein. If the polynucleotide encodes an antisense polynucleotide or a ribozyme, expression will produce the antisense polynucleotide or ribozyme. Thus in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Gene transfer techniques which target DNA directly the intestinal epithelium. Receptor-mediated gene transfer, for example, is accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, co-infection with adenovirus can be included to disrupt endosome function.

### Pharmaceutical Compositions and Formulations

The invention also encompasses the use of pharmaceutical compositions of the invention or salts thereof to practice the methods of the invention. Such a pharmaceutical composition may consist of at least one modulator composition of the invention or a salt thereof in a form suitable for administration to a subject, or the pharmaceutical composition may comprise at least one modulator composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Diagnostic Methods

The present invention provides a method to diagnose a subject having or at risk for developing a disease or disorder associated with intestinal microbiota. For example, in one embodiment, the method comprises using level of expression or activity of one or more components of the NLRP6 inflammasome, mucin secretion pathway, or autophagy pathway as diagnostic markers. In one embodiment, the method comprises detecting the presence of a genetic mutation in a nucleic acid encoding one or more components of the NLRP6 inflammasome, mucin secretion pathway, or autophagy pathway. In one embodiment, the method comprises evaluating the amount of mucin secretion, the integrity of a mucous layer, or the presence of mucin granules. For example, as described herein, deficiency in NLRP6 inflammasome-mediated mucin secretion is characterized by the presence of mucin granules within goblet cells that do not fuse with the apical membrane of the epithelium. Thus, in one embodiment, histological observation of such granules in goblet cells is used to diagnose a disease or disorder associated with intestinal microbiota.

In one embodiment, the method is used to diagnose a subject as having a disease or disorder associated with intestinal microbiota. In one embodiment, the method is used to diagnose a subject as being at risk for developing a disorder associated with intestinal microbiota. In one embodiment, the method is used to evaluate the effectiveness of a therapy for a disease or disorder associated with intestinal microbiota.

In one embodiment, the method comprises collecting a sample from a subject. Exemplary samples include, but are not limited to blood, urine, feces, sweat, bile, serum, plasma, tissue biopsy, and the like. For example, in one embodiment, the sample comprises a cell of the intestinal epithelium. In one embodiment, the sample comprises a goblet cell.

Methods for detecting a reduced expression or activity of one or more components of the NLRP6 inflammasome or autophagy pathway comprise any method that interrogates a gene or its products at either the nucleic acid or protein level. Such methods are well known in the art and include, but are not limited to, nucleic acid hybridization techniques, nucleic acid reverse transcription methods, and nucleic acid amplification methods, western blots, northern blots, southern blots, ELISA, immunoprecipitation, immunofluorescence, flow cytometry, immunocytochemistry. In particular embodiments, disrupted gene transcription is detected on a protein level using, for example, antibodies that are directed against specific proteins. These antibodies can be used in various methods such as Western blot, ELISA, immunoprecipitation, or immunocytochemistry techniques.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### NLRP6 Inflammasome Regulates the Intestinal Host-Microbial Interface by Orchestrating Goblet Cell Mucus Secretion

Microbial dysbiosis and the increased susceptibility to DSS-induced colitis in NLRP6 deficient mice suggest that NLRP6 may play an important role in intestinal barrier maintenance. The primary defense against microbial and pathogen penetration into the lamina propria is the single layer of epithelium cells and its associated protective mucus layer. Goblet cells (GC), specialized intestinal epithelial cells, produce and secrete mucins, predominantly Muc2, into the intestinal lumen, thereby forming the mucus layer (Tytgat et al., 1994, Gastroenterology, 107: 1352-1363). Muc2 biosynthesis involves protein dimerization in the ER, glycosylation in the Golgi apparatus, oligomerization and dense packing of these large net-like structures into secretory granules of the goblet cell (Ambort et al., 2012, Proceedings of the National Academy of Sciences of the United States of America, 109: 5645-5650). Mucin-containing granules are stored within a highly organized array of microtubules and intermediate filaments called the theca, which separates mucin granules from the rest of the cytoplasm and gives mature goblet cells their distinctive shape (Forstner, 1995, Annual Review of Physiology, 57: 585-605). Exocytosis of mucin occurs when apically oriented mucin granules fuse with the plasma membrane in a complex but not understood process (Ambort et al., 2012, Proceedings of the National Academy of Sciences of the United States of America, 109: 5645-5650; Forstner, 1995, Annual Review of Physiology, 57: 585-605). The resultant intestinal mucus layer consists of two stratified layers and plays a key role in the maintenance of intestinal homeostasis; it protects the epithelium from dehydration, physical abrasion, and commensal and invading microorganisms (Johansson et al., 2008, Proceedings of the National Academy of Sciences of the United States of America, 105: 15064-15069; Linden et al., 2008, Mucosal Immunology, 1: 183-197). In contrast to the loose matrix and microbiota containing outer mucus layer, the inner mucus layer composition is dense and devoid of the microbiota (Johansson et al., 2008, Proceedings of the National Academy of Sciences of the United States of America, 105: 15064-15069), and functions as a barrier, which serves to minimize microbial translocation and prevent excessive immune activation. Muc2-deficient mice, which lack a normal intestinal mucus layer, are more susceptible to intestinal inflammation and infection, stemming from heightened commensal or pathogenic microbial interaction with the epithelial layer (Gill et al., 2011, Cellular microbiology, 13: 660-669; Van der Sluis et al., 2008, Laboratory Investigation; a Journal of Technical Methods and Pathology, 88: 634-642; Van der Sluis et al., 2006, Gastroenterology, 131: 117-129). Muc2 deficiency leads to exacerbated disease by the attaching and effacing (A/E) pathogen, Citrobacter rodentium, characterized by an increased rate of pathogen colonization and an inability to clear pathogen burdens through increased mucus secretion (Bergstrom et al., 2010, PLoS Pathogens, 6: e1000902).

Mucus production by goblet cells of the large intestine serves as a crucial anti-microbial protective mechanism at the interface between the eukaryotic and prokaryotic cells of the mammalian intestinal ecosystem. However, the regulatory pathways involved in goblet cell-induced mucus secretion remain largely unknown. Here it is demonstrated that the NLRP6 inflammasome, a recently described regulator of colonic microbiota composition and bio-geographical distribution, is a critical orchestrator of goblet cell mucin granule exocytosis. NLRP6 deficiency leads to defective autophagy in goblet cells and abrogated mucin secretion into the large intestinal lumen. Consequently, NLRP6 inflammasome-deficient mice are unable to clear enteric pathogens from the mucosal surface, rendering them highly susceptible to persistent infection. The study described herein identifies the first innate immune regulatory pathway governing goblet cell mucus secretion, linking non-hematopoietic inflammasome signaling to autophagy and highlighting the goblet cell as a critical innate immune player in the control of intestinal host-microbial mutualism.

The materials and methods employed in the experiments are now described.

Mice

NLRP6^{-/-} (Elinav et al., 2011b, Cell, 145: 745-757), ASC^{-/-} (Sutterwala et al., 2006, Immunity, 24: 317-327), Casp1^{-/-} (Kuida et al., 1995, Science, 267: 2000-2003), Atg7^{+/-}, IL-1R^{-/-} and IL-18^{-/-} (Takeda et al., 1998, Immunity, 8: 383-390) mice were described in previous publications. All mice were backcrossed at least 10 times to C56B1/6. WT C56B1/6 mice were purchased from NCI. GFP-LC3 transgenic mice were obtained from Jackson laboratories and crossed with NLRP6^{-/-} mice. All mice were specific pathogen-free, maintained under a strict 12 h light cycle (lights on at 7:00 am and off at 7:00 pm), and given a regular chow diet (Harlan, diet #2018) ad libitum.

Immunohistochemistry

Paraffin embedded tissues either Bouins or Carnoys-fixed were deparaffinized and rehydrated. Antigen retrieval was performed in 10 mM citric acid pH 6.0 at 90-100° C. Immunostaining was carried out using antibodies against Tir, Clca3 (M-53, Santa Cruz), Muc2 (H-300, Santa Cruz), MPO (Ab-1, Thermo Scientific) and CD90.1 (eBioscience) antibody followed by incubation with an Alexa-conjugated secondary antibody (Invitrogen) or the FITC conjugated UEA-I lectin (EY laboratories). Tissues were mounted using ProLong Gold® Antifade (Molecular Probes/Invitrogen) that contains 4′,6′-diamidino-2-phenylindole (DAPI) for DNA staining.

In Situ Hybridization

Segments of the ascending colon were dissected and fixed in 4% paraformaldehyde in 1× PBS overnight at 4° C., washed in 70% ethanol and then paraffin-embedded. 7 mm tissue sections were soaked in xylene to remove paraffin and then post-fixed for 10 min. After washing with 1×PBS, sections were digested with 3 mg/ml proteinase K at room temperature for 20 min and washed in PBS again before acetylation with 0.25% acetic anhydride in 0.1M triethanolamine/0.9% NaCL (pH 8.0) for 10 min. Slides were then rinsed with 2×SSC followed by incubation in 0.66% N-ethylmaleimide for 30 min. After rinsing in 2×SSC, sections were dehydrated through graded ethanols, soaked in chloroform for 2 min, rehydrated to 95% ethanol and air-dried. Hybridization with ³⁵S-labeled cRNA probes (sense or antisense) composed of a 412 bp segment of the mouse NLRP6 gene (representing nucleotides 63 to 474 of the mRNA) was performed as described (Wysolmerski et al., 1998). Sections were then stained by the periodic acid-Schiff technique (with Alcian blue counterstain) to identify mucin-containing cells and air dried, followed by the application of photographic emulsion (Kodak NTB) and development after an exposure time of three weeks.

Transmission and Scanning Electron Microscopy

Mice were perfused via their left ventricles using 4% paraformaldehyde in PBS. Selected tissues were fixed in 2.5% glutaraldehyde in 0.1 M sodium cacodylate buffer pH 7.4 for 1-2 h. Samples were rinsed three times in sodium cacodylate buffer and post-fixed in 1% osmium tetroxide for 1 h, en bloc stained in 2% uranyl acetate in maleate buffer pH 5.2 for a further hour then rinsed, dehydrated, infiltrated with Epon812 resin, and baked overnight at 60° C. Hardened blocks were cut using a Leica UltraCut UCT. 60-nm-thick sections were collected and stained using 2% uranyl acetate and lead citrate. Samples were all viewed in an FEI Tencai Biotwin TEM at 80 kV. Images were taken using Morada CCD and iTEM (Olympus) software.

RNA Isolation and cDNA Synthesis

The terminal 2-3 mm of the colon were excised, immediately submerged in RNAlater™ (Qiagen) and stored at 4° C. overnight and then at −80° C. for subsequent RNA extraction. RNA was extracted using RNeasy Mini kit (Qiagen) according to the manufacturer's instructions. RNA concentration was determined using a NanoDrop ND-1000 (NanoDrop Technologies, Wilmington, Del., USA) and reverse transcription was performed with the Quantitect RT kit (Qiagen) using 1 μg RNA as template.

Real-Time Polymerase Chain Reaction

Real-time PCR was performed using Quantitect SYBR-Green Mastermix (Qiagen) and QuantiTect Relmβ, Reg3β and Reg3γ (Qiagen) in addition to NLRP6, IL-22, IL-18, Muc2, TFF-3, Muc1, Muc3, and Muc 4 (listed below). PCR was performed on an Opticon 2 (Bio-Rad) and cycles consisted of 95° C. for 15 min and 40 cycles of 94° C. for 15 s, 60° C. for 30 s and 72° C. for 30 s. Glyceraldehyde-phosphate-dehydrogenase (GAPDH) was used for normalization. The fold difference in expression was calculated as 2-ΔΔC(t).

NLRP6
(SEQ ID NO: 1)
F-CACACCCAGAATGAGACCAG
(SEQ ID NO: 2)
R-GTAGCCATAAGCAGCTCCCT
IL-22
(SEQ ID NO: 3)
F-GCAATCAGCTCAGCTCCTGT
(SEQ ID NO: 4)
R-CGCCTTGATCTCTCCACTCT
IL-18
(SEQ ID NO: 5)
F-CAGGCCTGACATCTTCTGCAA
(SEQ ID NO: 6)
R-TCTGACATGGCAGCCATTGT
Muc2
(SEQ ID NO: 7)
F-GCTGACGAGTGGTTGGTGAATG
(SEQ ID NO: 8)
R-GATGAGGTGGCAGACAGGAGAC
TFF3
(SEQ ID NO: 9)
F-CCTGGTTGCTGGGTCCTCTG
(SEQ ID NO: 10)
R-GCCACGGTTGTTACACTGCTC
Muc1
(SEQ ID NO: 11)
F-GCAGTCCTCAGTGGCACCTC
(SEQ ID NO: 12)
R-CACCGTGGGCTACTGGAGAG
Muc3
(SEQ ID NO: 13)
F-CGTGGTCAACTGCGAGAATGG
(SEQ ID NO: 14)
R-CGGCTCTATCTCTACGCTCTCC
Muc4
(SEQ ID NO: 15)
F-CAGCAGCCAGTGGGGACAG
(SEQ ID NO: 16)
R-CTCAGACACAGCCAGGGAACTC
GAPDH
(SEQ ID NO: 17)
F-ATTGTCAGCAATGCATCCTG
(SEQ ID NO: 18)
R-ATGGACTGTGGTCATGAGCC

Goblet Cell and Mucus Layer Preservation Ex Vivo

The terminal 5 mm of the colon were excised, immediately submerged in Ethanol-Carnoy's fixative at 4° C. for 2 hours and then placed into 100% ethanol. Fixed colon tissues were embedded in paraffin and cut into 5 μm sections. Tissues were stained with Alcian blue/PAS.

Western Blot

Colonic epithelial cells were isolated from the colon using an EDTA/PBS wash. Total cells were lysed with MP-40 and protease inhibitor cocktail (Roche Diagnostics). Membranes were probed with anti-LC3 (Novus Biologicals), anti-p62 (Sigma) and anti-actin then an anti-rabbit/goat-HRP antibody.

Bacterial Strains and Infection of Mice

Mice were infected by oral gavage with 0.1 mL of an overnight culture of LB containing approximately 1×10⁹ cfu of a kanamycin-resistant, luciferase-expressing derivative of C. rodentium DBS 100 (ICC180), and analyzed on day 15 post infection, unless otherwise stated.

Citrobacter rodentium CFU, Antibody Titers and Cytokine Determination

Whole mouse spleen and colon tissues were collected in 1 mL of sterile PBS supplemented with complete ethylenediaminetetracetic acid-free protease inhibitor cocktail (Roche Diagnostics) at a final concentration recommended by the manufacturer. Tissues were weighed, homogenized in a MixerMill 301 bead miller (Retche) for 2 minutes at room temperature. Tissue homogenates were serially diluted in PBS and plated on to LB kanamycin plates, incubated overnight at 37° C., and bacterial colonies were enumerated the following day, normalizing them to the tissue weight (per gram). C. rodentium colonies were clearly identified by kanamycin resistance and luciferase signal. Colon homogenates were centrifuged twice at 15,000 g for 20 min at 4° C. to remove cell debris, and the supernatants were aliquoted and stored at −80° C. ELISA plates were coated with whole C. rodentium, incubated with colonic or splenic lysates to determine IgA and IgG antibody titers, respectively. Cytokine levels in colon homogenates were determined with the BD Cytometric Bead Array Mouse Inflammation Kit (BD Biosciences), according to the manufacturer's recommendations, and normalized to tissue weight (per gram).

Bioluminescent Imaging (BLI) In Vivo and Ex Vivo

For in vivo BLI, mice were anesthetized with 1% isoflurane. Bioluminescence was quantified using Living Image software (Perkin Elmer), using 10 seconds exposure. For ex vivo BLI, colons were resected, extensively washed from all fecal matter, and immediately imaged. BLI was also used to visualize plated CFU dilutions.

Histopathological Scoring

Tissues were fixed in Bouin's medium and then placed into 70% ethanol. Fixed distal colon tissues were embedded in paraffin and cut into 5 μm sections. Tissues were stained with hematoxylin and eosin (H&E), using standard techniques by the Yale Research Histology Laboratory. Tissue sections were assessed for pathology in four regions: lumen, surface epithelium, mucosa and submucosa. Pathology in the lumen was based on the presence of necrotic epithelial cells (0=none; 1=scant; 2=moderate; 3=dense). The surface epithelium was scored for regenerative change (0=none; 1=mild; 2=moderate; 3=severe), desquamation (0=no change; 1=<10 epithelial cells shedding per lesion; 2=11-20 epithelial cells shedding per lesion) and ulceration (3=epithelial ulceration; 4=epithelial ulceration with severe crypt destruction). The mucosa was scored for hyperplasia (scored based on crypt length/high-power field averaged from four fields at 400× magnification where 0=<140 μm; 1=141-285 μm; 2=286-430 μm; 3=>431 μm) and goblet cell depletion (scored based on number of goblet cells/high-power field averaged from four fields at 400× magnification where 0=>50; 1=25-50; 2=10-25; 3=<10). Lastly, the submucosa was scored for edema (0=no change; 1=mild; 2=moderate; 3=profound). The maximum score that could result from this scoring is 21.

Statistical Analysis

Statistical significance was calculated by using a two-tailed Student's t-test unless otherwise stated, with assistance from GraphPad Prism Software Version 4.00 (GraphPad Software, San Diego, Calif., USA). If not otherwise specified statistical significance was given as ****p-value<0.0001; ***p-value<0.001; **p-value<0.01; *p-value<0.05; ns (not significant) p-value>0.05. The results are expressed as the mean value with standard error of the mean (SEM), unless otherwise indicated.

The results of the experiments are now described.

### NLRP6 Inflammasome Deficiency Impairs Host Mediated Enteric Pathogen Clearance

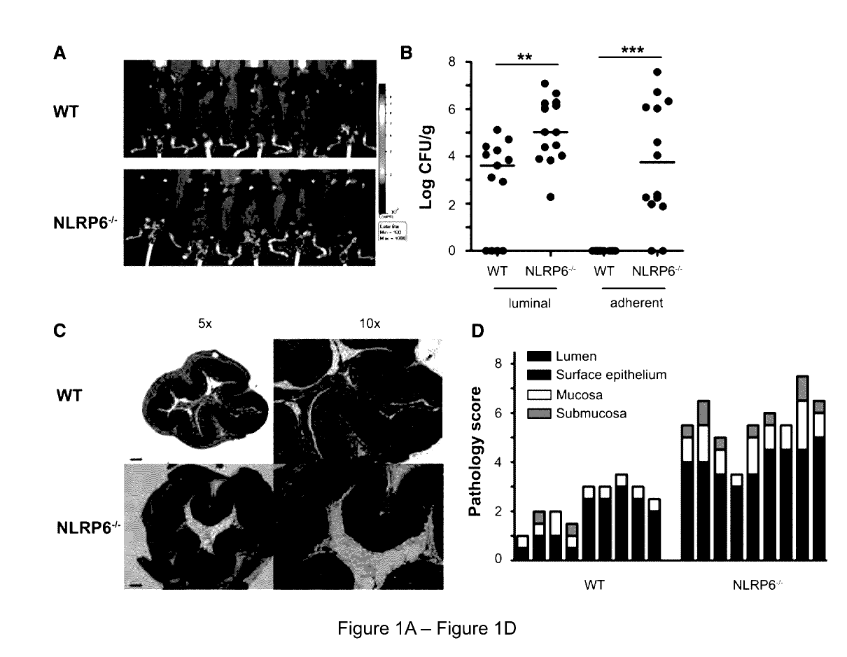

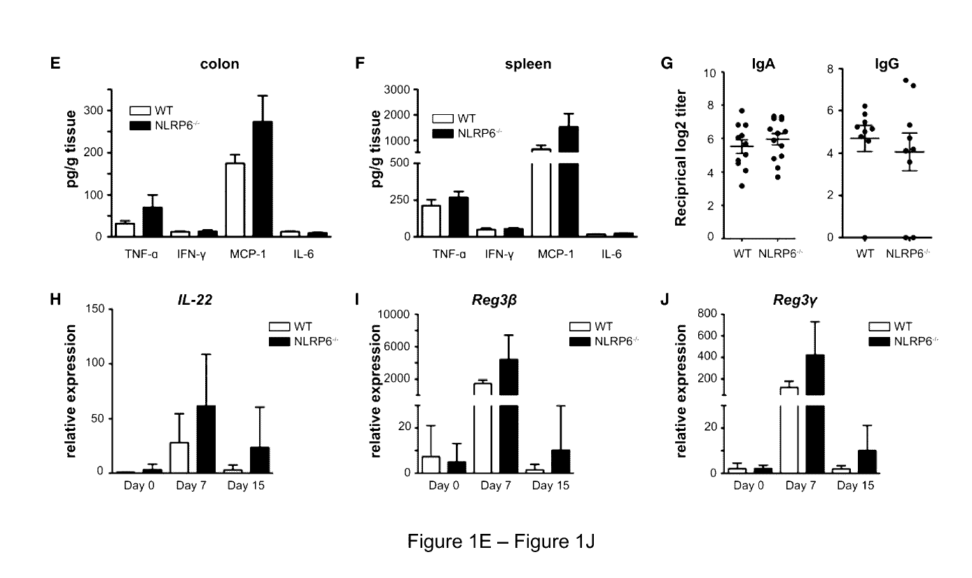

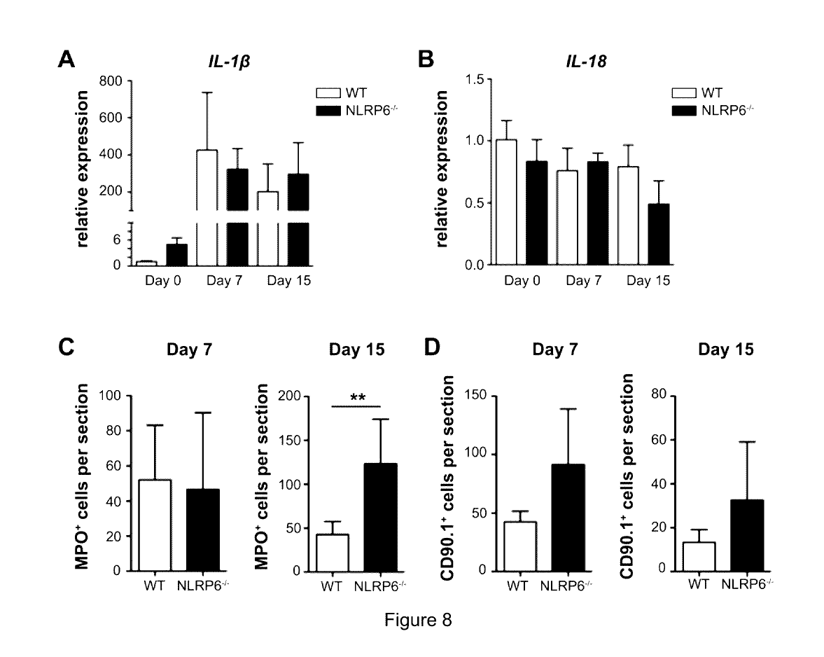

The NLRP6 regulates colonic microbial ecology, and NLRP6-deficient mice show altered microbial community composition, suggesting that NLPR6 inflammasome activity is involved in the maintenance of a stable community structure in the intestine (Elinav et al., 2011b, Cell, 145: 745-757). A major cause of microbial community disruption in the intestine is enteric infection. Mice infected with Citrobacter rodentium or Salmonella enterica undergo massive changes in microbiota composition (Lupp et al., 2007, Cell host & microbe, 2: 204; Stecher et al., 2007, PLoS biology, 5: 2177-2189). To analyze whether NLRP6 plays a role in host defense against enteric infections, the ability to clear C. rodentium by NLRP6-deficient mice was tested. A bioluminescent variant of C. rodentium was used, which allows for non-invasive in vivo monitoring of bacterial growth over the time course of the infection (Wiles et al., 2006, Infection and immunity, 74: 5391-5396). Remarkably, at day 9 p.i., Nlrp6^{-/-} mice were extensively colonized with C. rodentium when compared to WT mice (FIG. 1A). Total C. rodentium luminal (fecal matter only) and adherent (washed intestinal tissue only) burden of the large intestine were also significantly higher in Nlrp6^{-/-} mice at day 15 p.i. when compared to wild-type (WT) mice (FIG. 1B). Strikingly, at this late time-point 86% of the Nlrp6^{-/-} mice still had C. rodentium attached to the intestinal epithelium, in contrast to 0% of WT mice (FIG. 1B). This trend was reproducible regardless of the source of C57bl mice. Nlrp6^{-/-} mice also showed a significant increase in pathology in the distal colon at day 15 p.i. (FIG. 1C), confirming the high intestinal burdens of C. rodentium. This increase in pathology was characterized by greater submucosal edema, more extensive damage to the surface mucosa and ulceration, and extensive regions of mucosal hyperplasia (FIG. 1D). The increased C. rodentium burden and pathology at day 15 p.i. was not accompanied by decreased production of pro-inflammatory cytokines in the colon or spleen (FIG. 1E and FIG. 1F, respectively), C. rodentium-specific antibody profile (FIG. 1G), or impaired signaling through the IL-22 pathway and its related downstream anti-microbial peptides (FIG. 1H-FIG. 1J). Likewise, colonic IL-1β & IL-18 mRNA levels were similar in naïve and infected WT & NLRP6^{-/-} mice (FIG. 8A-FIG. 8B). Intestinal neutrophil and T cell numbers, as measured by myeloperoxidase and CD90.1 immunohistochemistry, respectively, were reactively elevated in NLRP6^{-/-} as compared to WT mice (FIG. 8C-FIG. 8D). This suggested that increased bacterial colonization in Nlrp6^{-/-} mice was not a result of an ineffective immune response to the pathogen, but rather by an alternate non-hematopietic cell-mediated mechanism.

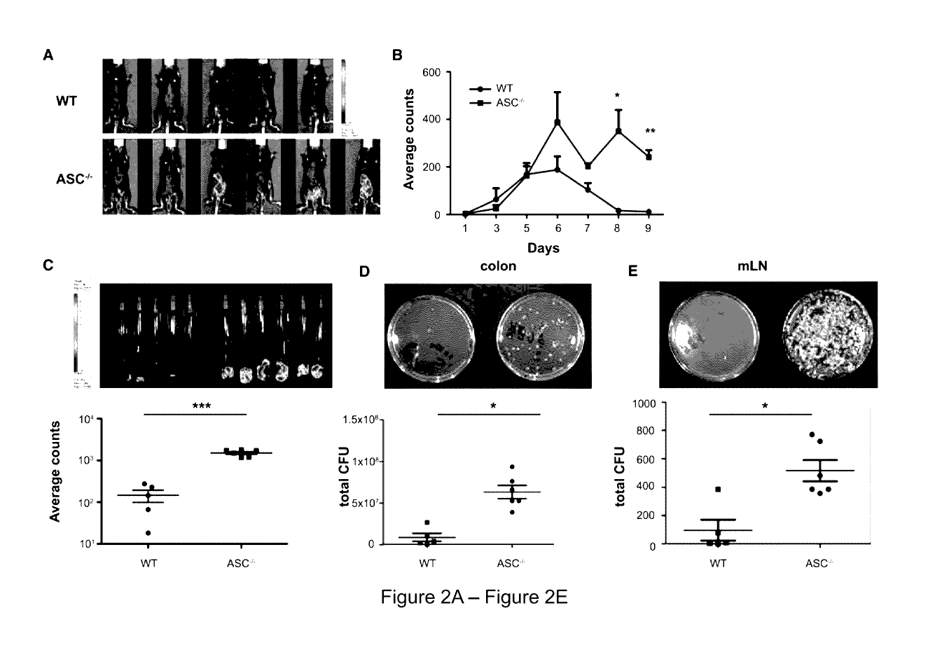

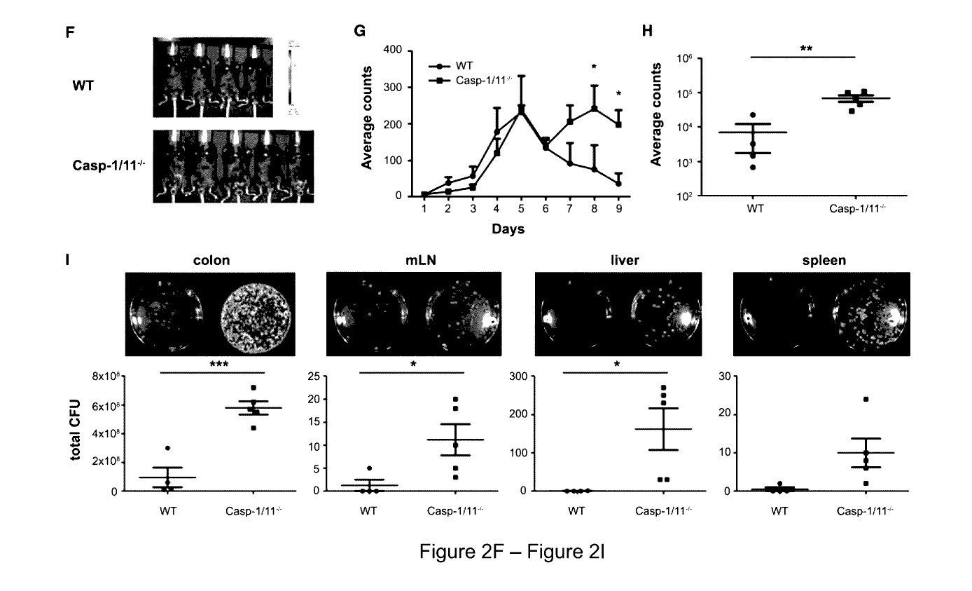

To determine whether an NLRP6 inflammasome was necessary for host defense to C. rodentium, mice deficient in ASC and caspase-1 were studied for their ability to clear C. rodentium infection. Like Nlrp6^{-/-} mice, Asc^{-/-} and Caspase-1/11^{-/-} mice were unable to clear C. rodentium from the colon and remained highly colonized while WT mice began to clear infection at day 9 p.i. (FIG. 2A-FIG. 2B, FIG. 2F-FIG. 2H). As a result, mice lacking any inflammasome component featured enhanced colonic and systemic colonization with C. rodentium (FIG. 2C-FIG. 2E, FIG. 2I). Collectively, these results suggested that NLRP6 inflammasome activation is pivotal for host defense against A/E pathogen infection.

### NLRP6 Contributes to Intestinal Homeostasis Through Regulation of Goblet Cell Function

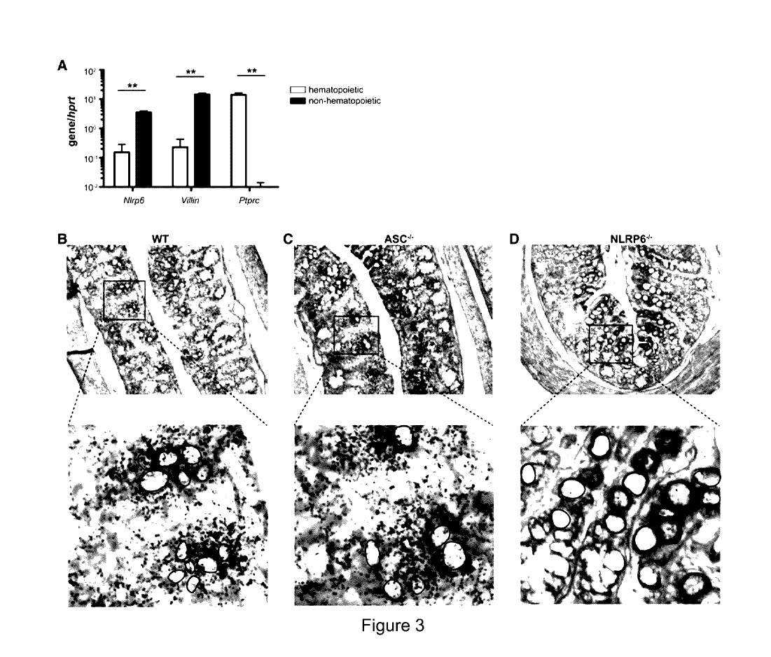

To understand the mechanism by which NLRP6 inflammasome activity contributes to host defense to enteric infection, it was sought to identify the cell type mediating this anti-pathogen response. It has been previously shown that NLRP6 is highly expressed within the non-hematopoietic intestinal compartment, especially within intestinal epithelial cells (Elinav et al., 2013a, Mucosal Immunology, 6: 4-13; Elinav et al., 2011b, Cell, 145: 745-757). This near-exclusive contribution of colonic epithelial cells to intestinal NLRP6 expression was maintained during Citrobacter infection, as measured by high purity sorting of epithelial and hematopoietic colon cells during day 10 of infection (FIG. 3A). However, these cells can be further divided based on morphologic and functional differences into various subsets, including enterocytes, goblet cells, Paneth cells and intestinal stem cells. To begin the investigation of the cellular source of NLRP6 activity, a series of in-situ hybridization studies were performed on colonic sections from WT, ASC^{-/-} and Nlrp6^{-/-} mice. NLRP6 was found to be highly expressed throughout the intestinal mucosa of WT mice, concentrated in the apical mucosal region (FIG. 3B, upper panel), specifically in goblet cells, seen as extensive probe binding in areas surrounding the theca containing mature mucin granules (FIG. 3B, lower panel). Intestines deficient in the adaptor protein, ASC, show similar NLRP6 expression and localization pattern (FIG. 3C), whereas Nlrp6^{-/-} mice remained negative to this staining (FIG. 3D). This expression pattern of NLRP6 suggested that NLRP6 contribute to mucosal defense by regulating goblet cell function and mucus production.

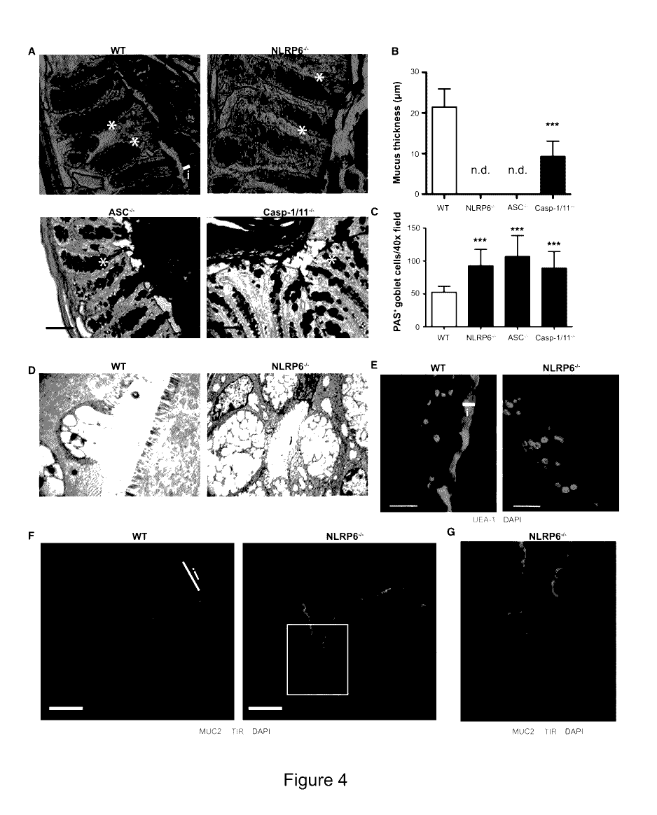

Mucus secretion is critically important in host defense against multiple enteric pathogens, including the A/E family of pathogens that adhere to the host surface epithelial layer where they perform their pathogenic functions (Gill et al., 2011, Cellular Microbiology, 13: 660-669). As an important line of defense, the host utilizes mucus secretion as a method to prevent attachment and remove the adherent load from the mucosal surface (Bergstrom et al., 2010, PLoS Pathogens, 6: e1000902). To explore whether defective goblet cell-mediated mucus secretion was indeed responsible for the enhanced susceptibility of NLRP6 inflammasome deficient mice to enteric infection, it was sought to characterize goblet cell function in Nlrp6^{-/-} inflammasome deficient and WT mice. Intriguingly, it was found that the intestinal epithelium of Nlpr6^{-/-}, Asc^{-/-}, and Caspase 1/11^{-/-} mice lack a thick continuous overlaying inner mucus layer (FIG. 4A and FIG. 4B, “i” inner mucus layer) and exhibit a marked goblet cell hyperplasia (FIG. 4A and FIG. 4C), suggesting a dramatic functional alteration in goblet cell mucus secretion in NLRP6 inflammasome deficient mice. Further exploring this deficiency, transmission electron microscopy was used to visualize the theca of goblet cells, which is normally packed with mucin granules. In WT mice, once the theca containing mucin granules reach the apical surface of the intestinal epithelium they fuse with the epithelium, releasing the stored mucins and associated proteins into the intestinal lumen (FIG. 4D, left panel). In contrast, the distal colon of Nlrp6^{-/-} mice featured increased accumulation of intracellular mucin granules and an apparent inability of these granules to fuse with the apical surface of the intestinal epithelium (FIG. 4D, right panel). Likewise, mucus staining with the lectin Ulex europaeus agglutinin I (UEA-1) revealed a lack of intact mucus layer and goblet cell hyperplasia in Nlrp6^{-/-} intestinal sections (FIG. 4E).

The abrogated mucus secretion in Nlrp6^{-/-} mice was expected to enable increased attachment of C. rodentium during infection. To address this, immunostaining for the C. rodentium-derived infection marker Tir (translocated intimin receptor) was performed on colon sections at day 7 p.i. as a measure of C. rodentium attachment to and infection of the intestinal epithelium. In the early stages of infection in WT mice, C. rodentium primarily infected the mucosal surface (Tir-positive) but did not invade the crypts (FIG. 4F). However, in Nlrp6^{-/-} mice, C. rodentium was dramatically more invasive, penetrated deeper into the crypts and was found more frequently associated with goblet cells (Muc2-positive, FIG. 4F-FIG. 4G). These results, in complete agreement with previous results featuring commensal bacteria in close approximation to the normally near-sterile crypt base (Elinav et al., 2011b, Cell, 145: 745-757), demonstrate that NLRP6 deficiency and resultant mucus alterations, result in abnormal microbial approximation to the host mucosal surface, leading to infectious, inflammatory, metabolic, and neoplastic consequences (Chen et al., 2011, Journal of Immunology, 186: 7187-7194; Elinav et al., 2011b, Cell, 145: 745-757; Normand et al., 2011, Proceedings of the National Academy of Sciences of the United States of America, 108: 9601-9606).

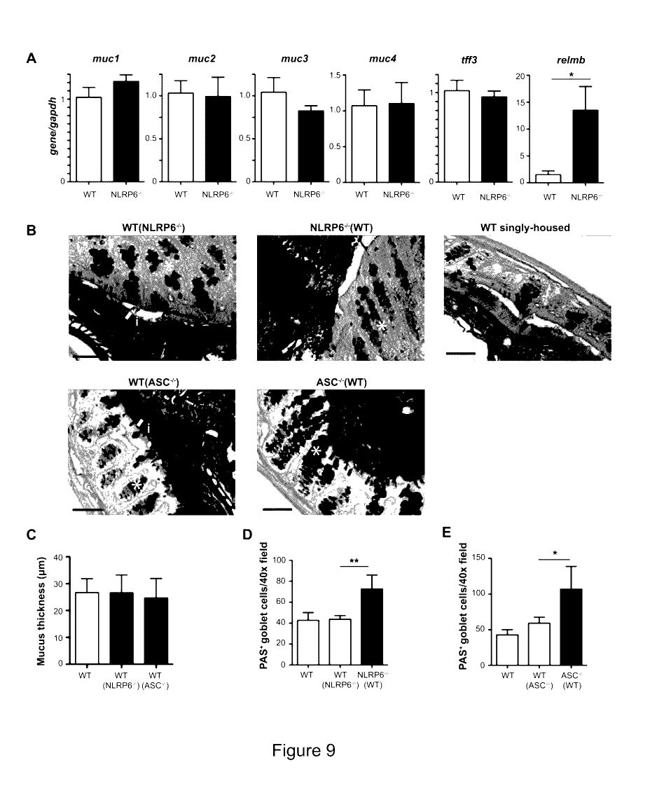

To further define this observed defect in mucus secretion, transcriptional regulation of goblet cell-specific proteins including the mucins, Muc1, Muc2, Muc3 and Muc4, intestinal trefoil factor 3 (TFF-3), and resistin-like molecule β (Relmβ) was assessed. These proteins have defined roles in intestinal homeostasis; Muc2 is a gel-forming mucin and the main component of the intestinal mucus layer (Johansson et al., 2008, Proceedings of the National Academy of Sciences of the United States of America, 105: 15064-15069), Muc1, Muc3 and Muc4 are surface bound mucins with roles in signaling and tumorigenesis, TFF3 synergizes with Muc2 to enhance the protective properties of the mucus layer (Van der Sluis et al., 2006, Gastroenterology, 131: 117-129), and Relmβ has an important role in innate immunity and host defense (Artis et al., 2004, Proceedings of the National Academy of Sciences of the United States of America, 101: 13596-13600; Nair et al., 2008, Journal of Immunology, 181: 4709-4715). No reduction was seen in any goblet cell specific protein transcript levels in Nlrp6^{-/-} mice (FIG. 9A). In fact, Relmβ expression was significantly elevated in these mice (FIG. 9A). This suggests that the deficiency in mucus production in Nlrp6^{-/-} mice is not due to reduced transcript production.

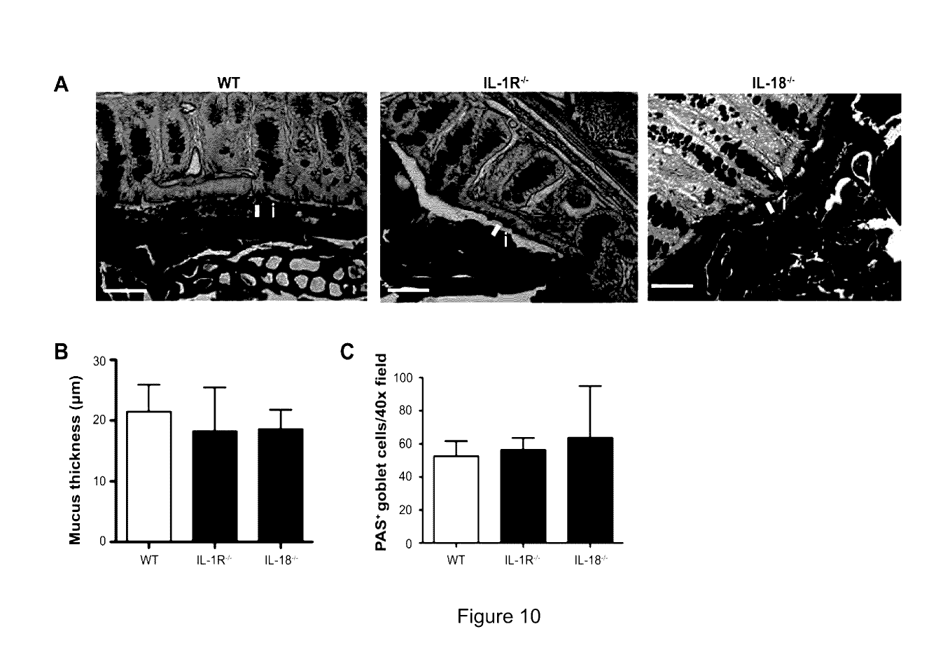

It has been recently demonstrated that NLRP6 inflammasome deficient mice feature a distinct microbiota configuration, which drives a context-specific susceptibility to intestinal auto-inflammation, non-alcoholic fatty liver disease, and colorectal cancer, through several microbial-induced mechanisms (Elinav et al., 2013a, Mucosal immunology, 6: 4-13; Elinav et al., 2011a, Immunity, 34: 665-679; Elinav et al., 2011b, Cell, 145: 745-757; Elinav et al., 2013b, Methods in Molecular Biology, 1040: 185-194; Henao-Mejia et al., 2012, Nature, 482: 179-185; Henao-Mejia et al., 2013a, Advances in Immunology, 117: 73-97; Henao-Mejia et al., 2013b, Journal of Autoimmunity, 46: 66-73; Hu et al., 2013, Proceedings of the National Academy of Sciences of the United States of America, 110: 9862-9867). To study whether the inflammasome deficient microbiota is responsible for the altered steady-state goblet cell phenotype, WT mice were cohoused with Nlrp6^{-/-} or Asc^{-/-} mice. This modality induces full microbiota configuration transfer into cohoused WT mice, allowing for direct assessment of the inflammasome deficient microbiota as compared to WT microbiota in singly housed WT mice. As is shown in FIG. 9B-FIG. 9E, cohoused WT mice featured a comparable mucus layer and goblet cell hyperplasia to that of singly-housed WT mice, ruling out a significant microbiota contribution to the observed goblet cell impairment in NLRP6 inflammasome deficient mice. Likewise, the mucus layer and goblet hyperplasia was normal in IL-1R^{-/-} and IL-18^{-/-} mice (FIG. 10), suggesting that the primary goblet cell defect in the absence of NLPR6 was mediated by IL-1- and IL-18-independent mechanisms.

### NLRP6 Regulates Goblet Cell Mucus Granule Secretion

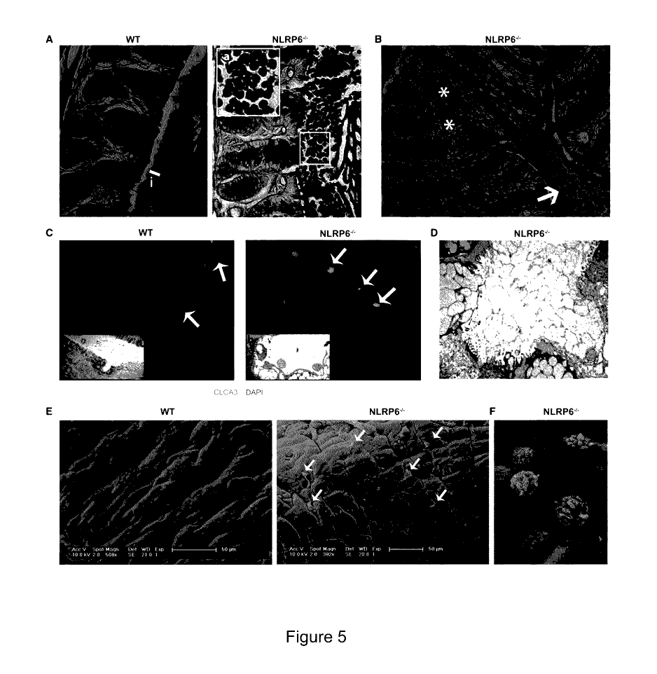

In addition to the lack of a continuous inner mucus layer in Nlrp6^{-/-} mice (FIG. 5A, “i”), mucin granule-like structures were also found in the lumen of Nlrp6^{-/-} mice (FIG. 5A, inset “a”). In several cases, these structures were densely packed in the intestinal lumen (FIG. 5B, arrow). They measured 6.28 μm±0.80 μm in diameter (100 granules measured) and were never found in WT mice. This width compares to the size of mucin-containing granules in mature goblet cells found in the mucosa, which measured 7.29 μm±2.18 μm in diameter (100 granules measured). In order to further confirm that these structures were mucin granules, immunoflouresence (FIG. 5C) and transmission electron microscopy (FIG. 5D) were used. Murine calcium-activated chloride channel family member 3 (mCLCA3, alias Gob-5) was previously identified as a protein exclusively associated with mucin granule membranes of intestinal goblet cells (Leverkoehne and Gruber, 2002, The Journal of Histochemistry and Cytochemistry: Official Journal of the Histochemistry Society, 50: 829-838). Immunofluorescence utilizing an anti-mCLCA3 antibody demonstrated punctate staining in the lumen of Nlrp6^{-/-} intestinal tissue (FIG. 5C) suggesting the presence of intact mucin granules in the lumen. In contrast, WT tissue showed punctate staining at the surface of the intestinal epithelium, where mucin granules fuse with the intestinal epithelium, and some diffuse staining in the lumen (FIG. 5C), as previously reported (Leverkoehne and Gruber, 2002, The Journal of Histochemistry and Cytochemistry: Official Journal of the Histochemistry Society, 50: 829-838). Transmission electron microscopy showed mucin granules protruding into the intestinal lumen with their membranes intact, with none of the granules found to fuse with or empty into the lumen. Furthermore, these intact membrane-bound structures were also present inside the lumen (FIG. 5D). Utilizing scanning electron microscopy, many protruding mucin granules were observed in the intestinal epithelium of Nlrp6^{-/-} mice (FIG. 5E, arrows), which were rarely seen in WT mice. Further, enlargement of the mucin granule protrusions clearly shows that each is made up of multiple granules (FIG. 5F). While not wishing to be bound by any particular theory, it is likely that these protruding mucin granules get sloughed off into the intestinal lumen via the shearing force of fecal matter passing through the intestine explaining their luminal presence in Nlrp6^{-/-} mice.

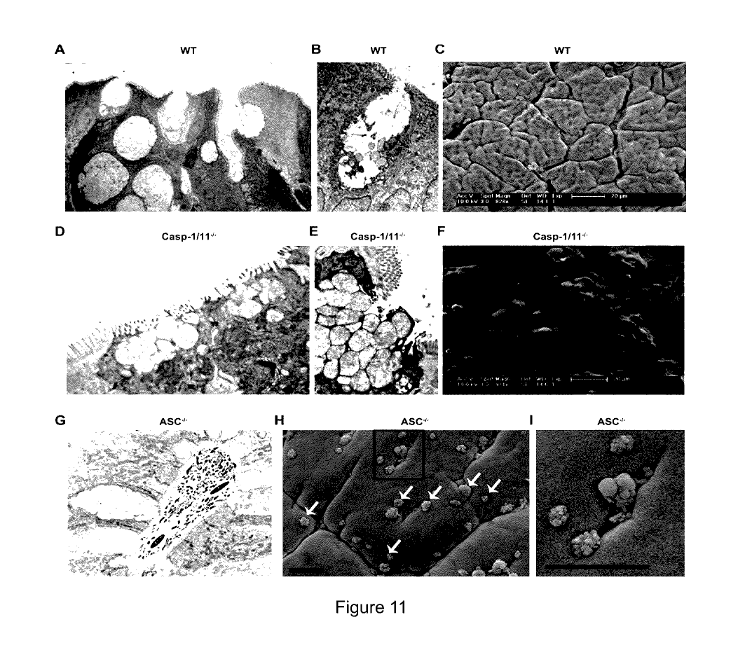

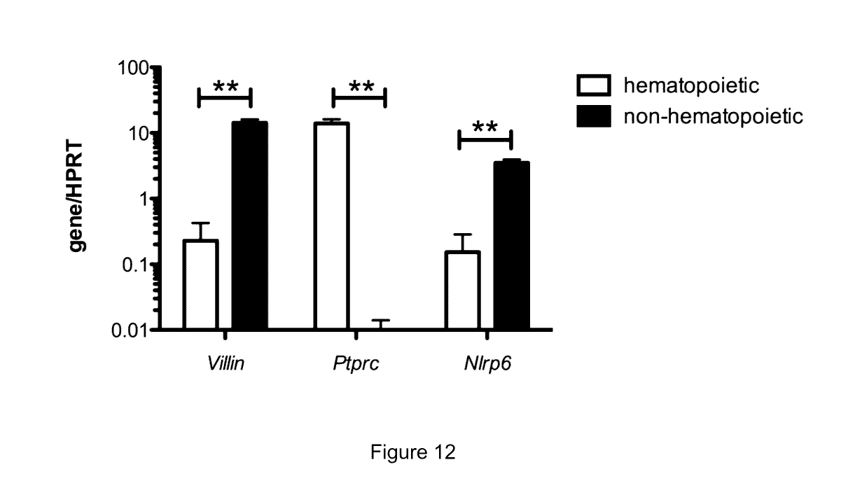

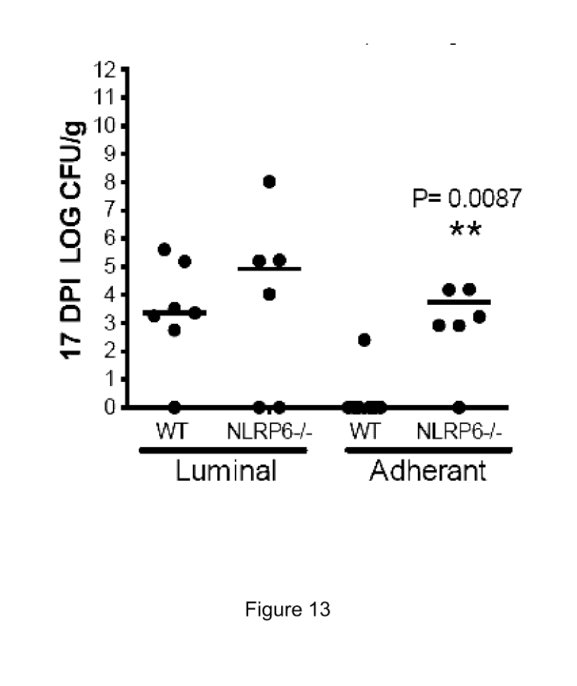

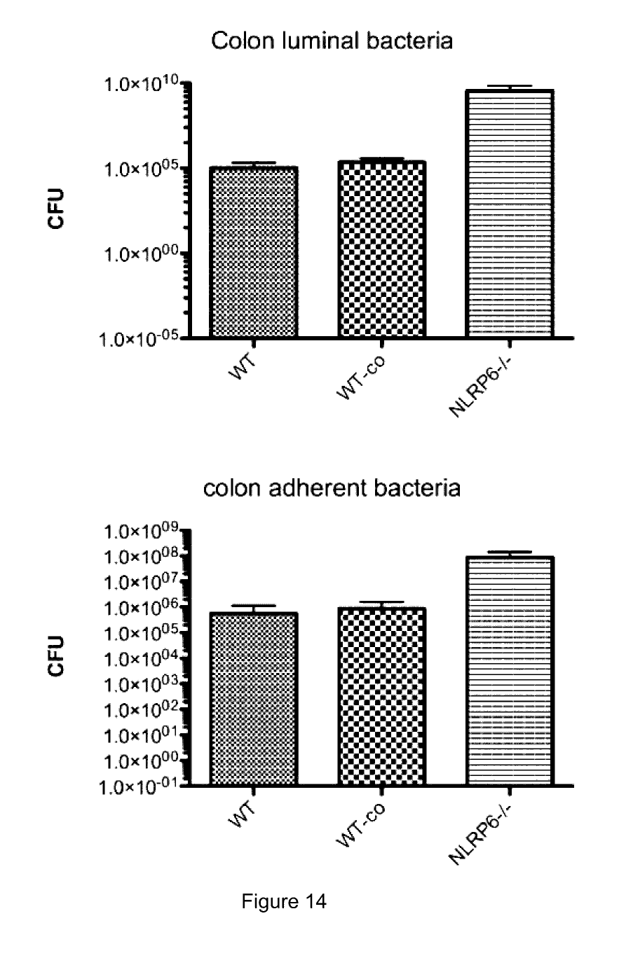

To determine if this novel function of NLRP6 requires recruitment of members of the classical inflammasome pathway to regulate mucus secretion, scanning (SEM) and transmissive (TEM) electron microscopy was utilized to characterize the intestinal mucus layer of caspase-1/11^{-/-} and ASC^{-/-} mice. In agreement with the observations above, goblet cells in Caspase-1/11^{-/-} and Asc^{-/-} mice were found to also feature goblet cells lacking mucus secretion. Caspase-1/11^{-/-} mice feature goblet cells with a weakly packed theca that upon fusion with the intestinal epithelium does not readily release contained mucin granules (FIG. 11D-FIG. 11E). Similar to Nlrp6^{-/-} mice, Asc^{-/-} mice show the accumulation of densely packed goblet cells with mucus granules protruding into the intestinal lumen without mucus secretion. Findings similar to the Nlrp6^{-/-} intestinal wall were evident with scanning electron microscopy in both the Caspase-1/11^{-/-} and Asc^{-/-} deficient intestinal epithelium (FIG. 11F and FIG. 11H), suggesting that both the NLRP6 sensor and assembly of the inflammasome complex are required for appropriate mucus granule fusion with the intestinal epithelium and subsequent mucus secretion.

### NLRP6 Inflammasome is Critical for Autophagy in Intestinal Epithelial Cells

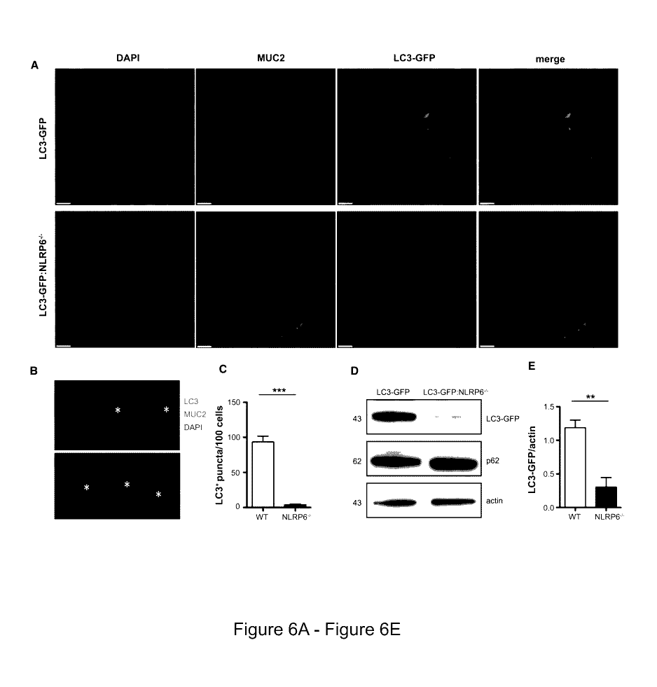

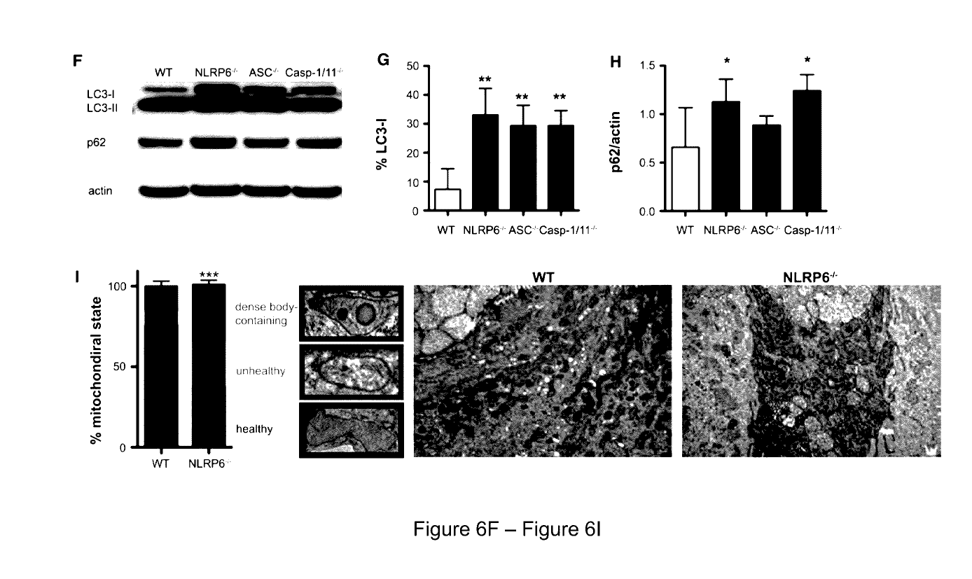

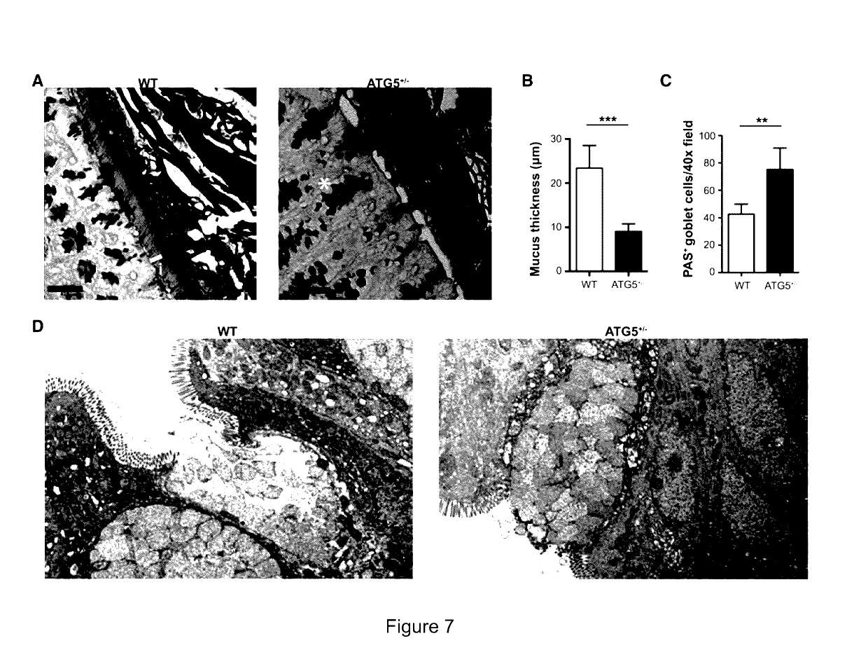

It was next sought to dissect the molecular pathways by which NLRP6 inflammasome signaling regulates goblet cell mucus secretion. Paneth cells are a small intestinal secretory epithelial cell subset that has functional importance in orchestration of the host-microbial interface by secretion of a variety of host-protective mediators. Paneth cells are normally not found within the large intestine, where the much less studied goblet cells are believed to mediate many similar host-protective secretory functions. In Paneth cells, autophagy has been shown to be critical for proper function of secretory pathways (Cadwell et al., 2008, Nature, 456: 259-263). Similar autophagy-mediated regulation of secretory pathways has been described in osteoclasts (DeSelm et al., 2011, Developmental cell, 21: 966-974) and mast cells (Ushio et al., 2011, The Journal of Allergy and Clinical Immunology, 127: 1267-1276). Furthermore, a recent proteomic study demonstrated the presence of an autophagy related protein, Atg5, in intestinal mucin granules (Rodriguez-Pineiro et al., 2012, Journal of Proteome Research, 11: 1879-1890). Moreover, mice with deletion of Atg7 in intestinal epithelial cells were recently found to feature enhanced susceptibility to C. rodentium infection (Inoue et al., 2012, Archives of Biochemistry and Biophysics, 521: 95-101). To determine if defective autophagy provided the mechanistic link between NLRP6 deficiency, goblet cell dysfunction, and enhanced enteric infection, NLRP6 deficient mice were crossbred with transgenic mice systemically expressing GFP fused to LC3. LC3 functions as a marker protein for autophagosomes (Mizushima et al., 2004, Molecular Biology of the Cell, 15: 1101-1111). During the formation of the autophagosome, the unconjugated cytosolic form of LC3 (called LC3-I) is converted to the phosphatidylethanolamine-conjugated (lipidated) form (called LC3-II) and incorporated to the membrane that is visible as discrete puncta using immunofluorescence analysis (Choi et al., 2013, The New England journal of medicine, 368: 1845-1846). In WT mice the LC3-GFP signal had a characteristic punctate staining indicative of the formation of autophagosomes (FIG. 6A). This LC3-GFP autophagosome staining was also localized within goblet cells (cells both Muc2- and GFP-positive, FIG. 6B). Strikingly, in NLRP6 deficient intestinal tissue, the LC3-GFP signal was absent (FIG. 6A and FIG. 6C). NLRP6 deficiency led to reduced levels of the LC3-GFP protein and an accumulation of p62 in isolated intestinal epithelial cells (FIG. 6D and FIG. 6E), indicative of diminished autophagosome formation. Endogenous LC3-I and LC3-II levels were also severely altered in Nlrp6^{-/-}, ASC^{-/-} and Casp-1/11^{-/-} mice in intestinal epithelial cells, featuring an elevated LC3-I/LC3-II ratio and accumulation of P62 (FIG. 6F-FIG. 6H). An accumulation of degenerating mitochondria, described as unhealthy lacking intact cristae and containing dense inclusion bodies of proteins, in NLRP6 deficient intestinal epithelium (FIG. 6I) further supported a defect in autophagy processes. Altogether, these results suggest that NLRP6 deficiency mediates profound autophagy impairment in goblet cells that, like in the functionally correlative Paneth cell, result in secretion alterations that lead to significant impairment in colonic host-microbial interactions. To definitely establish the link between inflammasome signaling and autophagy in mediating the goblet cell phenotype, ATG5^{+/-} mice were examined for goblet cell abnormalities. Remarkably, even partial deficiency of autophagy signaling (the homozygous mice are embryonically lethal) fully recapitulated the phenotype of mucus layer impairment, goblet cell hyperplasia, and secretory defects (FIG. 7A-FIG. 7D), substantiating the role of autophagy downstream of inflammasome signaling as a driver of goblet cell secretory function.

### NLRP6 Inflammasome-Mediated Mucin Granulin Exocytosis in Goblet Cells

This report represents the first described mechanism regulating mucin granule exocytosis by goblet cells in the large intestine, being mediated by the NLRP6 inflammasome. NLRP6 control of mucus secretion directly affects its ability to regulate intestinal and microbial homeostasis while creating a protective niche from enteric pathogens. Genetic deletion of NLRP6 and key components of the inflammasome signaling pathway, caspase-1 and ASC, leads to abrogated mucus secretion characterized by protruding mucin granules, that rather than fusing into the apical basement membrane and releasing their content, are sloughed off into the intestinal lumen in their entirety. It is demonstrated herein that NLRP6 is important in maintaining autophagy in the intestinal epithelium, a process previously shown to be critically important in intestinal granule exocytosis pathway.

It is shown herein that NLRP6 is highly expressed in the intestinal epithelium, specifically locating to apical regions surrounding the theca of mature goblet cells. No evidence of NLRP6 mRNA expression was found in the submucosal colonic region, including myofibroblasts (Normand et al., 2011, Proceedings of the National Academy of Sciences of the United States of America, 108: 9601-9606). Inflammasome signaling has classically been shown to mediate its immune functions through the production of pro-inflammatory cytokines, although there is recent supporting evidence that inflammasome function is also important in the biological function of a cell beyond IL-1β and IL-18 production. As an example, caspase-1/inflammasome signaling is essential in adipocyte differentiation and influencing insulin resistance in these cells (Stienstra et al., 2010, Cell Metabolism, 12: 593-605). Indeed, the data described herein point towards an IL-1- & IL-18-independent goblet cell intrinsic function of inflammasomes in regulating granule secretion. Nevertheless, both cytokines may still play key roles in the orchestration of multiple host-microbiota and inflammatory protective mucosal responses that may integrate with the cytokine-independent inflammasome roles described herein in shaping the host responses to its environment. The exact cell and context-specific roles of IL-1 and IL-18 in contributing to the overall roles mediated by intestinal inflammasomes thus merits further studies.

As of yet there have been only very few studies exploring the immune pathways that regulate mucus secretion (Songhet et al., 2011, PloS One, 6: e22459). Here, it is shown that NLRP6 is essential for baseline mucus secretion in both healthy and disease states, making it the first innate immune pathway to be implicated in regulating mucus secretion. The lack of mucus secretion and inability to form an adherent, continuous inner mucus layer would allow for close microbe-epithelium interactions in NLRP6 deficient mice, and provides an explanation to previously described observations that the dysbiotic microbiota in Nlrp6^{-/-} mice is intimately associated with the mucosa (Elinav et al., 2011b, Cell, 145: 745-757). This impaired host-microbial interface leads to context-dependent consequences that may include transcriptional epithelial cell reprogramming of CCL5 (Elinav et al., 2011b, Cell, 145: 745-757), influx of bacterial products into the portal circulation upon dietary induction of the metabolic syndrome (Henao-Mejia et al., 2012, Nature, 482: 179-185), and promotion of the IL-6 signaling pathway during inflammation-induced cancer (Hu et al., 2013, Proceedings of the National Academy of Sciences of the United States of America, 110: 9862-9867). As such, the combination of environment (mediating compositional and functional microbial alterations) and genetics (mediating mucus barrier defects through NLRP6 inflammasome deficiency), jointly drive compound ‘multi-factorial’ phenotypes such as colonic auto-inflammation, non-alcoholic steatohepatitis (NASH), and inflammation induced cancer (Chen et al., 2011, Journal of Immunology, 186: 7187-7194; Henao-Mejia et al., 2012, Nature, 482: 179-185; Normand et al., 2011, Proceedings of the National Academy of Sciences of the United States of America, 108: 9601-9606). The same alteration in the host-microbial interface may alternatively result in exacerbated infection when a pathogen, such as C. rodentum or its human correlate Enteropathogenic E. Coli, are introduced into the ecosystem. Therefore, a unified model is proposed explaining how host genetic variability (manifested as susceptibility traits in some individuals) coupled with distinct environmental insults may result in seemingly unrelated and variable phenotypic consequences. In human inflammatory bowel disease, as one example, such a model may explain the wide variability in clinical manifestations, even in the lifespan of individual patients, as a variety of intestinal and extra-intestinal auto-inflammatory manifestations, susceptibility to certain infections and a tendency for neoplastic transformation (Grivennikov et al., 2010, Cell, 140: 883-899).

Autophagy has been characterized as being crucial in maintaining the integrity of the Paneth cell granule exocytosis pathway (Cadwell et al., 2008, Nature, 456: 259-263). Deficiency in Atg16L1 led to decreased number and disorganized granules, decreased lysozyme secretion, intact granules present in the crypt lumen and an abundance of degenerating mitochondria. Likewise as shown herein, formation of autophagosomes in the intestinal epithelium, including within goblet cells, could be visualized. Further, it is demonstrated that NLRP6 deficient epithelium lacked visible autophagosome formation and an altered LC3I/II ratio. This suggests that the activity of the NLRP6 inflammasome is critical for autophagy induction and activity in the intestinal epithelium. Corresponding to a reduction in the activity of autophagy in the intestine of Nlrp6^{-/-} mice, there was an accumulation of p62 and an abundance of degenerating mitochondria, both targets of autophagy for degradation. Given the important function of autophagy in numerous secretory pathways (Cadwell et al., 2008, Nature, 456: 259-263; DeSelm et al., 2011, Developmental Cell, 21: 966-974; Ushio et al., 2011, The Journal of Allergy and Clinical Immunology, 127: 1267-1276) it is likely that the mechanism whereby NLRP6 deficiency leads to defective mucus granule exocytosis is by inhibiting the autophagic processes required for proper secretion of mucus granules. Such autophagy-induced regulation of goblet cell secretory functions was recently demonstrated to involve downstream reactive oxygen species signaling (Patel et al., 2013, The EMBO Journal).

Colonizing the outer mucus layer and penetrating the inner mucus layer is a key step in the pathogenesis of C. rodentium and is likely achieved by the production of virulence factors with mucinase activity (Bergstrom et al., 2010, PLoS pathogens, 6: e1000902). Further, goblet cell-driven mucus secretion has been shown to be critical in resolving C. rodentium infection by dissociating adherent C. rodentium from the intestinal mucosa (Bergstrom et al., 2008, Infection and immunity, 76: 796-811; Bergstrom et al., 2010, PLoS Pathogens, 6: e1000902). Likewise, in the present study, increased susceptibility to C. rodentium in Nlrp6^{-/-} mice is a consequence of the lack of an inner mucus layer and abrogated mucus secretion in the NLRP6 deficient mucosa. Further, NLRP6-mediated defense against this mucosal pathogen is dependent on inflammasome assembly, as deficiency in ASC and caspase-1 all resulted in increased C. rodentium burdens late in infection. Notably, other NLRP6 regulatory effects may contribute to containment of intestinal infection, such as those mediated by regulation of microbiota composition, recently highlighted to participate in C. rodentium clearance (Kamada et al., 2012, Science, 336: 1325-1329).

A recent study has shown increased resistance of Nlrp6^{-/-} mice to systemically administered bacterial pathogens, including Listeria monocytogenes, Salmonella Typhimurium and Escherichia coli (Anand et al., 2012, Nature, 488: 389-393). These results probably stem from differences in systemic versus local host related mechanisms of innate immune protection against invading pathogens. In a systemic bacterial infection, myeloid cells in circulation would be the primary responders to infection whereas in an intestinal bacterial infection epithelial cells would be involved in pathogen detection. It is not without precedence that inflammasome sensors have seemingly opposing function depending on the cell type involved, with important differences in hematopoietic cells versus non-hematopoietic cells for the NLRP6 inflammasome characterized (Anand et al., 2012, Nature, 488: 389-393; Chen et al., 2011, Journal of Immunology, 186: 7187-7194). Notably, the alteration in the mucosal anti-pathogenic immune response may be accompanied by a compensatory hyperactive systemic immune response, providing yet another example of the plasticity and rapid adoptability of the seemingly ‘primitive’ innate immune arm (Slack et al., 2009, Science, 325: 617-620).

The present study reveals the importance of the NLRP6 inflammasome in mucin-granule exocytosis, the first showing the relevance of inflammasome signaling in initiation of autophagy and maintaining goblet cell function. It suggests that goblet cells, previously regarded as passive contributors to the formation of the biophysical protective mucosal layers, may be actually active, regulatory hubs integrating signals from the host and its environment as an integral component of the innate immune response. Further mechanistic studies to assess the ligands for the NLRP6 inflammasome and how it may coordinate autophagy and the mucin-granule exocytosis pathway are of significant interest, as they impact greatly on host microbial interactions at mucosal interfaces.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for treating a disease or disorder associated with intestinal microbiota, the composition comprising a modulator of intestinal epithelium mucin secretion.

2. The composition of claim 1, wherein the modulator of intestinal epithelium mucin secretion is a modulator of at least one component of the NLRP6 inflammasome.

3. The composition of claim 1, wherein the modulator of intestinal epithelium mucin secretion is a modulator of at least one component of the autophagy pathway.

4. The composition of claim 1, wherein the modulator is at least one of the group consisting of a chemical compound, a protein, a peptide, a peptidomemetic, an antibody, a ribozyme, a small molecule chemical compound, a nucleic acid, a vector, an antisense nucleic acid molecule.

5. The composition of claim 1, wherein the modulator of intestinal epithelium mucin secretion is a modulator of goblet cell mucin secretion.

6. The composition of claim 1, wherein the disease or disorder is at least one selected from the group consisting of a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, non-alcoholic fatty liver disease (NAFLD), and non-alcoholic steatohepatitis (NASH).

7. The composition of claim 1, wherein the modulator of intestinal epithelium mucin secretion increases intestinal epithelium mucin secretion.

8. The composition of claim 1, wherein the modulator is a natural ligand expressed by at least one member of the intestinal microbiota.

9. A method of treating a disease or disorder associated with intestinal microbiota, the method comprising increasing intestinal epithelium mucin secretion in a subject.

10. The method of claim 9, wherein the method comprises administering to the subject a modulator of intestinal epithelium mucin secretion.

11. The method of claim 10, wherein the modulator of intestinal epithelium mucin secretion is a modulator of at least one component of the NLRP6 inflammasome.

12. The method of claim 10, wherein the modulator of intestinal epithelium mucin secretion is a modulator of at least one component of the autophagy pathway.

13. The method of claim 10, wherein the modulator is at least one of the group consisting of a chemical compound, a protein, a peptide, a peptidomemetic, an antibody, a ribozyme, a small molecule chemical compound, a nucleic acid, a vector, an antisense nucleic acid molecule.

14. The method of claim 10, wherein the disease or disorder is at least one selected from the group consisting of a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, non-alcoholic fatty liver disease (NAFLD), and non-alcoholic steatohepatitis (NASH).

15. The method of claim 10, wherein the method comprises administering a modulator of intestinal epithelium mucin secretion to a goblet cell of the subject.


SEQUENCE LISTING (18 sequences)

SEQ ID NO: 1 - DNA, 20 bp, Artificial Sequence
  Chemically synthesized
cacacccagaatgagaccag

SEQ ID NO: 2 - DNA, 20 bp, Artificial Sequence
  Chemically synthesized
gtagccataagcagctccct

SEQ ID NO: 3 - DNA, 20 bp, Artificial Sequence
  Chemically synthesized
gcaatcagctcagctcctgt

SEQ ID NO: 4 - DNA, 20 bp, Artificial Sequence
  Chemically synthesized
cgccttgatctctccactct

SEQ ID NO: 5 - DNA, 21 bp, Artificial Sequence
  Chemically synthesized
caggcctgacatcttctgcaa

SEQ ID NO: 6 - DNA, 20 bp, Artificial Sequence
  Chemically synthesized
tctgacatggcagccattgt

SEQ ID NO: 7 - DNA, 22 bp, Artificial Sequence
  Chemically synthesized
gctgacgagtggttggtgaatg

SEQ ID NO: 8 - DNA, 22 bp, Artificial Sequence
  Chemically synthesized
gatgaggtggcagacaggagac

SEQ ID NO: 9 - DNA, 20 bp, Artificial Sequence
  Chemically synthesized
cctggttgctgggtcctctg

SEQ ID NO: 10 - DNA, 21 bp, Artificial Sequence
  Chemically synthesized
gccacggttgttacactgctc

SEQ ID NO: 11 - DNA, 20 bp, Artificial Sequence
  Chemically synthesized
gcagtcctcagtggcacctc

SEQ ID NO: 12 - DNA, 20 bp, Artificial Sequence
  Chemically synthesized
caccgtgggctactggagag

SEQ ID NO: 13 - DNA, 21 bp, Artificial Sequence
  Chemically synthesized
cgtggtcaactgcgagaatgg

SEQ ID NO: 14 - DNA, 22 bp, Artificial Sequence
  Chemically synthesized
cggctctatctctacgctctcc

SEQ ID NO: 15 - DNA, 19 bp, Artificial Sequence
  Chemically synthesized
cagcagccagtggggacag

SEQ ID NO: 16 - DNA, 22 bp, Artificial Sequence
  Chemically synthesized
ctcagacacagccagggaactc

SEQ ID NO: 17 - DNA, 20 bp, Artificial Sequence
  Chemically synthesized
attgtcagcaatgcatcctg

SEQ ID NO: 18 - DNA, 20 bp, Artificial Sequence
  Chemically synthesized
atggactgtggtcatgagcc